(19) United States
(12) Patent Application Publication
(10) Pub. No.: US 2011/0206612 A1
(43) Pub. Date: Aug. 25, 2011

(54) NOVEL SCREENS TO IDENTIFY AGENTS THAT MODULATE RETINAL BLOOD VESSEL FUNCTION AND PERICYTE FUNCTION AND DIAGNOSTIC AND THERAPEUTIC APPLICATION THEREFOR

(75) Inventors: DONALD KEITH MARTIN, KILLARA (AU); NATALYA MARKHOTINA, WOOLLOOMOOLOO (AU)

(73) Assignee: UNIVERSITY OF TECHNOLOGY, SYDNEY, BROADWAY (AU)

(21) Appl. No.: 13/092,842

(22) Filed: Apr. 22, 2011

Related U.S. Application Data
(63) Continuation of application No. 12/115,489, filed on May 5, 2008, now abandoned.
Application No. 12/115,489 is a continuation of application No. 10/517,078, filed on Jun. 2, 2005, now abandoned.

(30) Foreign Application Priority Data
Jun. 7, 2002 (AU) PS 2847

(51) Int. Cl. A61K 49/00; C12Q 1/02; A61K 38/02; A61K 31/196; A61K 31/192; A61P 27/02; A61P 9/00

(52) U.S. Cl. 424/9.1; 435/29; 514/20.8; 514/567; 514/570

(57) ABSTRACT

The present invention provides methods for determining or identifying compounds that modulate the function of an isolated retinal pericyte or blood vessel, wherein a change in the contractile state of said pericyte or blood vessel is determined in the presence of a test compound, said change indicating that the test compound modulates the function of pericytes and/or blood vessels.

### CROSS-REFERENCE TO RELATED APPLICATIONS

This application is a continuation of application Ser. No. 12/115,489, filed May 5, 2008, which is a continuation of application Ser. No. 10/517,078, filed Jun. 2, 2005, now abandoned, which is U.S. National Phase of International Application No. PCT/AU03/00706, filed Jun. 6, 2003, which claims priority to Australian Patent Application No. PS 2847, filed Jun. 7, 2002, all of which are incorporated by reference in their entireties.

### BACKGROUND OF THE INVENTION

1. Field of the Invention

The present invention is in the field of drug screening, such as, for example agents that modulate cellular contractility or blood flow. More particularly, the present invention relates to novel screens for agents that modulate retinal blood vessel function, such as, for example, permeability, integrity, and contractility. Further the present invention relates to novel screens for agents that modulate pericyte function, such as, for example, the contraction of pericytes, cell growth, differentiation, ion channel conductivity, neurotransmitter release, or gene transcription. The agents identified using such screens are useful for the diagnosis or therapeutic treatment of disorders involving impaired retinal blood vessel function or pericyte function, such as for example retinal oedema, glaucoma, retinopathy, or retinal neovascularisation in human or animal subjects.

2. Description of the Related Art

The retina of the eye is a seven-layered structure involved in signal transduction. In most primates the retina is about 200-250 micrometers thick. The retina vasculature is relatively sparse and in most mammalian retinas the main arteries divide to form just two or three capillary beds. Capillaries in the retina comprise pericyte cells which extend processes down the long axis of, as well as around, the capillaries. Pericytes are contractile cells the contractions of which most likely regulate blood flow through the retinal microvasculature. This regulation of blood flow is mediated by the adhesion plaques, gap junctions and pericytic processes that communicate between the pericyte and the endothelial cells of the retina. Pericytic processes “attach” to several endothelial cells via so-called “peg and socket” junctions. Thus, the pericyte is in a unique position to influence flux across the microvasculature by affecting the “open” or “shut” state of endothelial intercellular junctions mechanically or by influencing endothelial cell contractile elements and/or endothelial cell transcytotic processes via humoral, ionic or other signal. Functional evidence for the contractile ability of the pericytes was demonstrated using cells grown on a silicone rubber substrate, using a modification of the method of Harris et al (1980) Science Vol 208.

It is thought that microvascular pericytes are physiological regulators of fluid, nutrient, protein and hormone movement across the microcirculatory endothelial barrier and that the pathophysiology of several disease states including diabetes mellitus might be comprehended more fully when viewed in terms of alterations in a pericyte function. Diseases of the eye that can involve vascular complications include glaucoma, corneal angiogenesis, retinopathy of prematurity, and diabetic retinopathy. For example, impaired blood flow at and around the optic nerve head may be a major cause of complications associated with glaucoma.

In diabetic retinopathy, blood vessels (arterioles and capillaries) throughout the retina become damaged or blocked resulting in a lack of blood supply to small areas of the retina. Diabetes can thereby lead to changes in the permeability of the retinal vasculature. In more advanced cases of diabetic retinopathy, retinal neovascularisation can lead to leakage of blood into the retina, and retinal detachment, with consequent loss of vision. If diabetic eye disease is left untreated, it can lead to serious visual impairment or blindness.

Approximately 25% of diabetics have some degree of diabetic retinopathy. Diabetic retinopathy occurs in both Type I diabetics and Type II diabetics. Nearly all Type I diabetics will have evidence of diabetic retinopathy after twenty years and up to 21% of all Type II diabetics have retinopathy when they are first diagnosed with diabetes. Because Type II diabetes is often not diagnosed until the patient has had the disease for many years, diabetic retinopathy may be present in Type II patients at the time diabetes is discovered. In fact, many patients first learn that they have diabetes when their ophthalmologist finds diabetic retinopathy on a routine eye examination.

The diagnosis of retinopathy currently requires the invasive use of an ophthalmoscope and/or fluorescein angiography to assess the function of the capillaries of the retina. The procedure can also provide some confirmation of a preliminary diagnosis of diabetes. Unfortunately, there is currently no easily performed test for susceptibility of retinal vessels to develop permeability defects.

### SUMMARY OF THE INVENTION

The present invention provides methods for determining or identifying compounds that modulate the function of a blood vessel in an isolated retina, wherein a change in the contractile state of a blood vessel in an isolated retina is determined in the presence of a test compound, said change indicating that the test compound modulates the function of the blood vessel.

The present invention also provides methods for determining or identifying compounds that modulate pericyte function, wherein a change in the contractile state of a pericyte is determined in the presence of a test compound, said change indicating that the test compound modulates pericyte function.

The compounds identified in the screens of the invention are those small molecules, peptides, proteins, hormones, nucleic acids, organic or inorganic chemical compounds etc that are agonists of retinal blood vessel function or pericyte function. Such compounds can modify ie, protagonise or antagonise contraction of a blood vessel in an isolated retina or a pericyte.

As used herein “an agonist” is a compound that can act on a pericyte cell or blood vessel to produce a physiological reaction, more particularly to modify contraction of a blood vessel or a pericyte.

By “protagonise contraction” is meant that the compound enhances the contraction of the blood vessel or pericyte, causing them to contract more fully or to a greater extent, or for a longer duration or more rapidly.

By “antagonise contraction” is meant that the compound reduces the extent of contraction of the blood vessel or pericyte, thereby causing the blood vessel or pericyte to relax, to relax more fully, or that the duration of any contraction is reduced or that the period of contraction is shortened or the relaxation phase of the cell is enhanced or prolonged.

By protagonising or antagonising the contraction of the blood vessel or pericyte, the contractile state of the blood vessel or pericyte is modified.

For example, an agonist can cause a relaxed pericyte to contract or a contracted pericyte to relax. Similarly, a partially relaxed or partially contracted pericyte can be caused to relax more fully or for a prolonged period, or alternatively, to contract more fully or for a prolonged period.

In another example, a relaxed retinal blood vessel is caused to contract or a contracted vessel is caused to relax. A partially relaxed or partially contracted blood vessel can be caused to relax more fully or for a prolonged period or alternatively to contract more fully or for a prolonged period.

As used herein, the term “contractile state” shall be taken to mean the extent to which a blood vessel or pericyte is contracted. Accordingly, with regard to a pericyte, a reduced contractile state is one wherein the pericyte is more turgid or expanded or relaxed, or alternatively less contracted or slower to contract or relaxed for a longer duration. Conversely, an enhanced or increased contractile state is one wherein the pericyte is more contracted or constricted or contracts more rapidly or remains contracts for a longer period.

With regard to a blood vessel in an isolated retina, a reduced contractile state is one wherein the blood vessel is more turgid or expanded or enlarged or relaxed, or dilated, or alternatively less contracted or slower to contract or relaxed for a longer duration. Conversely, an enhanced or increased contractile state is one wherein the blood vessel is more contracted or constricted or contracts more rapidly or remains contracted for a longer period.

The agonists identified in the novel screens of the invention are particularly useful in the diagnosis of impaired pericyte function, or impaired retinal blood vessel function, such as, for example, in cases of glaucoma, corneal angiogenesis, retinopathy of prematurity, or diabetic retinopathy.

By way of exemplification, the inventors have identified several lead compounds that modify the contractile state of a pericyte. The inventors have also identified several lead compounds that modify the contractile state of a blood vessel in an isolated retina.

In one embodiment, such compounds are capable of enlarging the retinal blood vessels and thereby increase blood flow to the retina. In an alternate embodiment, such compounds are capable of restricting the retinal blood vessels and thereby decreasing blood flow to the retina. Further, the inventors have developed a diagnostic test based on the potential for the retinal blood vessels including small capillary blood vessels to have an impaired function in controlling blood flow in the eye. The effects of the agonists can be observed using recognised means according to a skilled person in the art. In a particularly preferred embodiment the diagnostic test enables a practitioner to assess the risk of a subject developing glaucoma, corneal angiogenesis, retinopathy of prematurity, or diabetic retinopathy.

Accordingly, a second aspect of the invention provides a method of diagnosing impaired retinal blood vessel function in a subject comprising administering to the subject an amount of a pharmaceutically acceptable compound that modulates blood vessel function under conditions sufficient to modify the contractile state of a blood vessel, wherein said compound is identified by a method comprising determining a change in the contractile state of a blood vessel in an isolated retina in the presence of said compound, wherein said change is indicative that the compound modulates blood vessel function, and detecting a change in the contractile state of the subject's retinal vessels. Preferably, the change in the contractile state of the subject's retinal blood vessel(s) is determined by a comparison with the change in contractile state of a retinal blood vessel of a healthy subject.

In a related aspect, the invention provides a method of diagnosing impaired pericyte function in a subject comprising administering to the subject an amount of a pharmaceutically acceptable compound that modulates pericyte function under conditions sufficient to modify the contractile state of the subject's pericyte, wherein said compound is identified by a method comprising determining a change in the contractile state of a pericyte in the presence of said compound, wherein said change is indicative that said compound modulates pericyte function and detecting a change in the contractile state of the subject's pericytes.

In a related embodiment, the present invention provides a method of diagnosing retinal blood vessel damage in a subject, comprising administering to the subject an amount of a pharmaceutically acceptable compound that modulates the contractile state of a pericyte, under conditions sufficient to modify the contractile state of the subject's pericyte, wherein said compound is identified by a method comprising determining a change in the contractile state of a pericyte in the presence of said compound, wherein said change is indicative that the compound modulates pericyte function; and detecting dilation or constriction of a capillary in the retina of the subject, wherein a slow or unsubstantial dilation or constriction of the retinal blood vessel indicates retinal blood vessel damage.

By “pharmaceutically acceptable” is meant considered to be safe for administration to the eye of an animal.

Preferably, the change in the contractile state of the subject's retinal blood vessel is determined by a comparison with the change in contractile state of a retinal blood vessel of a healthy subject.

In one embodiment, the retinal blood vessel is a retinal capillary.

The agonists identified in the novel screens of the invention are also particularly useful in the therapeutic treatment of impaired pericyte function, or impaired retinal blood vessel function, such as, for example, in cases of glaucoma, corneal angiogenesis, retinopathy of prematurity, or diabetic retinopathy.

In particular, protagonists of pericyte function are useful for the therapy of a condition wherein pericytes exhibit a reduced or impaired contractile force or strength, or contract more slowly or for a shorter duration than the pericytes of a healthy subject. The application of such protagonist compounds may be indicated in cases where there is an oversupply of blood to the retina, such as, for example, in cases of diabetic retinopathy.

Similarly, compounds identified as protagonists of the function of a blood vessel in an isolated retina are useful for the therapy of a condition wherein retinal blood vessels exhibit a reduced or impaired contractile form or strength or contract more slowly or for a shorter duration than the retinal blood vessels of a healthy subject. The application of such protagonist compounds may also be indicated in cases where there is an oversupply of blood to the retina or where the retinal vessels are damaged or leaky, such as, for example in cases of diabetic retinopathy.

Conversely, antagonists of blood vessel or pericyte function identified according to the inventive method are useful for the treatment or therapy of a condition wherein the retinal blood vessels or pericytes are excessively stimulated to contract, or remain in a contracted state, or exhibit a reduced ability to relax, or relax fully, or where there is a reduced supply of blood to the vessels of the retina.

Surgical intervention may also involve the use of such compounds.

Accordingly, in a third aspect, the present invention provides for the use of a compound that modulates retinal blood vessel function in the preparation of a medicament for the treatment of impaired retinal blood vessel function in a subject, said compound being identified by a method comprising determining or identifying a change in the contractile state of a blood vessel in an isolated retina in the presence of said compound, wherein said change is indicative that the compound modulates retinal blood vessel function.

In a related aspect, the present invention provides for the use of a compound that modulates pericyte function in the preparation of a medicament for the treatment of impaired pericyte function in a subject, said compound being identified by a method comprising determining a change in the contractile state of a pericyte in the presence of said compound, wherein said change is indicative that the compound modulates pericyte function.

In a fourth aspect, the present invention provides methods for treating a subject having impaired retinal blood vessel function in a subject comprising administering to the subject an amount of a pharmaceutical composition comprising a compound that modulates retinal blood vessel function and a pharmaceutically acceptable carrier, wherein said compound is identified by a method comprising determining a change in the contractile state of a blood vessel in an isolated retina in the presence of said compound, wherein said change is indicative that the compound modulates retinal blood vessel function.

In a related aspect, there is provided a method for treating a subject having impaired pericyte function comprising administering to the subject an amount of a pharmaceutical composition comprising a compound that modulates pericyte function and a pharmaceutically acceptable carrier, diluent or excipient, wherein said compound is identified by a method comprising determining a change in the contractile state of a pericyte in the presence of said compound, wherein said change is indicative that the compound modulates pericyte function.

### BRIEF DESCRIPTION OF THE DRAWINGS

FIG. 1 shows a phase contrast image of pericytes (after 24 hr growth) contacting a silicone substrate, obtained using a videocamera (SONY SSC-DC30P) and framegrabber (DT3100) attached to an inverted microscope (NIKON TE200). Contraction of the pericytes was observed as wrinkles induced in the silicone substrate. Wrinkles are indicated by the arrows. A wrinkle was easily distinguished from the cell membrane by (i) a variable thickness along its length and (ii) marked changes in phase along its length. All of the pericytes in this field exhibit wrinkling of the silicone substrate.

FIG. 2 shows a phase contrast image of pericytes (after 24 hr growth) contacting a silicone substrate, obtained using a videocamera (SONY SSC-DC30P) and framegrabber (DT3100) attached to an inverted microscope (NIKON TE200). Contraction of the pericytes was observed as wrinkles induced in the silicone substrate. Wrinkles are indicated by the arrows. A wrinkle was easily distinguished from the cell membrane by (i) a variable thickness along its length and (ii) marked changes in phase along its length. All of the pericytes in this field exhibit wrinkling of the silicone substrate.

FIG. 3 is a graphical representation showing the dose-dependent effect of norepinephrine (NE) on the contraction of retinal pericytes. The concentration of NE is indicated on the x-axis. Contractility index, as determined from the number of silicone wrinkles for pericyte cultures, is indicated on the abscissa. NE intensified the number of silicone wrinkles in a contraction-dependent manner, inducing development of the contractile tone of the pericytes. NE at concentrations greater than 10⁻⁶M caused contraction, with maximum enhancement of contractile state of the pericytes at 10⁻⁴ M NE. NE induced pericyte contraction of silicone substrate in a dose-dependent manner (EC₅₀=8 μM.

FIG. 4 is a graph of the effect of Pituitary Adenylate Cyclase Activating Peptide (PACAP) on the contractility of pericytes. The EC₅₀ concentration is 3 nM, for these data averaged from 6 cells. A contractility index of less than 100 indicates that the pericyte had a relaxed response to the drug.

FIG. 5 is a graph of the effect of Vasoactive Intestinal Peptide (VIP) on the contractility of pericytes. The EC₅₀ concentration is 48 nM, for these data averaged from 3 cells. A contractility index of less than 100 indicates that the pericyte had a relaxed response to the drug.

FIG. 6 is a graph of the effect of different concentrations of Rp-cAMPS on the relaxation of pericytes that had been stimulated by PACAP (10⁻⁸ M). The numbers of pericytes included in the sample-size for each concentration is included in each bar.

FIG. 7 is a graph of the effect of inhibiting phospholipase C on the PACAP-induced relaxation of pericytes. The conditions indicated by the bars are as follows: U32 U73122 alone, UP=U73122 in the presence of PACAP, UPR=U73122 in the presence of both PACAP and Rp-cAMPS.

FIG. 8 is a graph of the effect of different concentrations of N-phenylanthranilic acid on the relaxation of pericytes. The data are averaged from a sample of 7 pericytes. A contractility index of less than 100 indicates that the pericyte had relaxed in response to the drug.

FIG. 9 is a graph of the effect of different concentrations of flufenamic acid on the relaxation of pericytes. The data are averaged from a sample of 4 pericytes. A contractility index of less than 100 indicates that the pericyte had relaxed in response to the drug.

FIG. 10 is a graph of the effect of different concentrations of R-flurbiprofen on the relaxation of pericytes. The data are averaged from a sample of 8 pericytes. A contractility index of less than 100 indicates that the pericyte had relaxed in response to the drug.

FIG. 11 is a graph of the effect of different concentrations (.mu.M) of aspirin on pericyte cells.

FIG. 12 is a graph of the effect of 0.3 μM aspirin on blood vessels in an isolated retina over time.

FIG. 13 is a graph of the effect of 1 μM aspirin on blood vessels in an isolated retina over time.

FIG. 14 is a graph of the effect of 3 μM aspirin on blood vessels in an isolated retina over time.

FIG. 15 is a graph of the effect of flurbiprofen at varying concentrations on pericyte cells for varying times.

FIG. 16 is a graph of the effect of 0.3 μM R-flurbiprofen on blood vessels in an isolated retina over time.

FIG. 17 is a graph of the effect of 0.3 μM R-flurbiprofen on blood vessels in an isolated retina over time.

FIG. 18 is a graph of the effect of 1 μM R-flurbiprofen on blood vessels in an isolated retina over time.

FIG. 19 is a graph of the effect of different concentrations of R-flurbiprofen on pericyte cells.

FIG. 20 is a graph of the effect of 1 μM S-flurbiprofen on blood vessels in an isolated retina over time.

FIG. 21 is a graph of the effect of 3 μM S-flurbiprofen on blood vessels in an isolated retina over time.

FIG. 22 is a graph of the effect of 10 μM S-flurbiprofen on blood vesssels in an isolated retina over time.

FIG. 23 is a graph of 0.3 μM aspirin on pericyte cells over time.

FIG. 24 is a graph of the effect of 10 μM aspirin on pericyte cells over time.

FIG. 25 is a graph of the effect of 1 μM aspirin on pericytes over time.

FIG. 26 is a graph of the effect of 3 μM aspirin on pericytes over time.

FIG. 27 is a graph of the effect of varying concentrations of aspirin on blood vessels in an isolated retina after 10 minutes.

FIG. 28 is a graph of the effect of 0.1 μM R-flurbiprofen on blood vessels in an isolated retina over time.

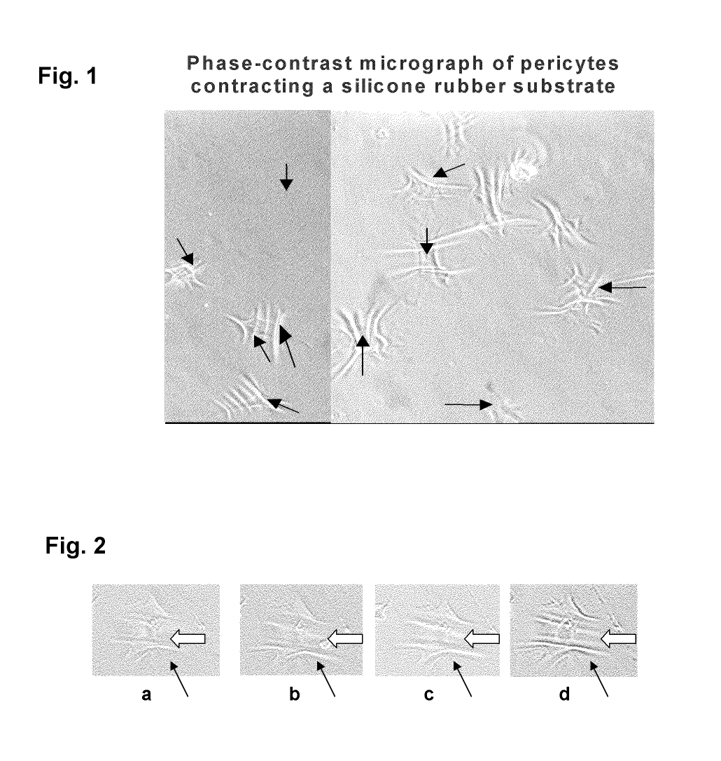

### DETAILED DESCRIPTION OF THE PREFERRED EMBODIMENT

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Pericyte Screening Assay

The present invention provides methods for determining or identifying compounds that modulate pericyte function, wherein a change in the contractile state of a pericyte is determined in the presence of a test compound, said change indicating that the test compound modulates pericyte function. In one embodiment, the invention provides a method for determining or identifying a compound that modulates the contractile state of a pericyte comprising contacting a pericyte with a test compound and determining a change in the contractile state of said pericyte, wherein said change indicates that the compound modulates the contractile state of the pericyte.

In one embodiment, the method comprises incubating the pericyte in the presence of a test compound.

Preferably, the contractile state of the pericyte is determined before the compound is contacted with the pericyte, thereby facilitating a determination of the change in contractile state of the pericyte. Alternatively, in certain preferred assay formats, the pericyte generally assumes a contracted state in the absence of a test compound, in which case relaxation of the pericyte or a reduced contractile state will be readily detectable.

Any art recognised means can be used to determine the contractile state of a pericyte, such as, for example, visual detection, second messenger assay involving a determination of cAMP levels or Ca²⁺ efflux, FACS analysis, ion channel activity, determining cellular permeability, or by determining a force that the cell applies to a surface with which the cell is in contact and the like.

The contractile state of a pericyte is preferably detected by visual means, such as, for example, employing a microscope or similar analytical tool wherein the size or volume of the cell in vivo or in vitro or in situ and change thereto can be detected. Preferably, the inventive method employs a technique for visualization, maintenance or culture of pericytes such that their contraction or relaxation can be readily detected, and subjected to quantitation.

In another embodiment, the contractile state of a pericyte is detected by a non-visual means.

The inventive method clearly encompasses the sequential contact of pericytes with a range of different compounds, that may be protagonistic or antagonistic towards each other. Cells may be washed between contacts to remove the residual first compound or reduce the concentration of said compound to level that is too low to exert an effect on the contraction of the pericyte.

In an alternative embodiment, the method further comprises contacting the pericyte with a second test compound without washing the cells between contacts with said first and second test compounds. In such cases, a second compound that is antagonistic towards the first compound will reverse the effects of the first compound if present at a sufficiently high concentration. Alternatively, where the second compound is a protagonist of the first compound, the contractile state of the pericyte may be enhanced step wise (ie enhanced contraction or enhanced relaxation).

In one embodiment, the pericyte is in physical contact with a resilient support. In alternative embodiment, the pericyte is not in physical contact with a resilient support. In one embodiment the pericyte is in aqueous solution.

In a particularly preferred embodiment, the contractile state of a pericyte is determined by the growth of a pericyte on a medium having a resilient or flexible support that is capable of being distorted when the pericyte is in contact therewith and capable of being distorted when the contractile state of the pericytes in contact therewith is modified.

The term “distort” and “distortion” as used herein refers to a change from the natural, previous, normal, or original shape or condition of a cell or support.

In one embodiment, the present invention provides a method of identifying a compound that modulates the contractile state of a pericyte comprising

(i) providing a pericyte cell in physical contact with a resilient support under conditions sufficient for a pericyte contraction to distort said resilient support;

(ii) contacting a test compound with said pericyte cell; and

(iii) determining a distortion in said resilient support, wherein said distortion indicates that said compound modulates the contractile state of the pericyte.

As used herein “physical contact” means sufficiently adherent for a change in the contractile state of the pericyte to produce a distortion in the support. Preferably, the cell is fixed to the resilient support.

Preferably, the resilient support is a resilient sheet. The term “resilient” refers to the characteristic of being capable of withstanding permanent distortion, deformation or rupture, or being capable of recovering from or adjusting to change or treatment. In the present context, the resilient support need only be sufficiently resilient to deform or be distorted during contraction or relaxation of a pericyte that is adhered thereto and to assume its normal appearance when the contractile state of the pericyte is modified. Highly elastic materials are not required for this purpose, since the extent of deformation of the support required is small.

Preferably the support is constructed of a glass.

In another embodiment, the support is constructed of non-glass material. In one embodiment, the support is constructed of a polymeric material, such as, for example, a cross-linked polymer. Preferably, a polymeric material comprises an elastic polymer or polymer film with elastic properties. In one embodiment, the support is a visco-elastic material. In one embodiment, the support is constructed from transparent material. In an alternate embodiment the support is constructed from a non-transparent material or semi-transparent material.

Mixed supports of glass and polymeric materials are particularly preferred.

Preferably, the support comprises glass having a cross-linked polymer coating to which the pericytes are attached or fixed or otherwise in physical contact with. In one embodiment the support has a cross-linked silicone fluid layer.

Preferably the support is level or planar to facilitate the adherence or culture of a monolayer or bilayer of cells thereto. Use of an uneven support may, in certain circumstances hinder quantitation because of the greater difficulty of correlating a single distortion with a single cell's contractile state. By “level or planar” means sufficiently even to permit the growth or culture or adherence of an even layer of cells in connection therewith, such as, for example an approximate monolayer of cells or an approximate bilayer of cells.

In an alternate embodiment, it may be suitable to use a support that is not level or planar.

When healthy pericytes are cultured on very thin sheets of cross-linked polymer, the traction forces that the cells exert on the thin sheet distort the polymer layer. Accordingly, contacting a test compound with said pericyte cell may cause further distortion of the polymer layer, by increasing contraction of the cell or decreasing contraction of the cell wherein said further distortion indicates that said compound modulates the contractile state of the pericyte.

Preferably, the distortion of the polymer layer is visualised as elastic distortion or wrinkling of the polymer layer.

In one embodiment the contractile state of a cell is determined by measuring or otherwise determining the change in size or volume of the cell in vivo or in vitro or in situ. For example, the contractile index of a cell can be readily determined by counting the number of wrinkles or distortions of the support.

The inventive method clearly encompasses the sequential incubation of pericytes with a range of different compounds, that may be antagonistic towards each other.

In one embodiment, the method further comprises:

washing the pericyte with a suitable buffer or aqueous solvent that is not damaging to the integrity or contractile function of the cell for a time and under conditions sufficient to remove the compound or reduce its activity to a level that does not affect pericyte contraction;

(ii) optionally determining a distortion in said resilient support;

(iii) contacting the pericyte with a second test compound; and

(iv) determining a distortion in said resilient support, wherein said distortion indicates that said compound modulates the contractile state of the pericyte.

In an alternative embodiment, the method further comprises contacting the pericyte with a second test compound without washing the pericyte with a suitable buffer or aqueous solvent under conditions sufficient to remove the compound or reduce its activity to a level that does not affect pericyte contraction, wherein a further distortion in the resilient support indicates that the compound modulates the contractile state of the pericyte.

In either embodiment, if the second distortion is opposed to the distortion obtained for the first test compound, then the second compound is an antagonist of the first compound. If the second distortion amplifies the distortion achieved for the first test compound, then the second compound is a protagonist of the first compound. For example, a first test compound may induce relaxation of a pericyte, thereby reducing wrinkling of the resilient support, whereas a second compound may induce pericyte contraction, thereby increasing the number of wrinkles in the resilient support. On the other hand, if both the first and second compounds produce the same effect, the number of wrinkles in the resilient support may be enhanced in a step wise manner between the addition of the first and second compounds (for agonists of pericyte function) or reduced in a step wise manner (for antagonists of pericyte function). All such possibilities are encompassed by the invention.

Preferably, the method further comprises comparing a visualised distortion of a cell from a subject with a control cell which can be treated (by contacting with a candidate compound) or untreated. In one embodiment, the control cell is a healthy cell. In an alternate embodiment, the control cell is an unhealthy cell such as for example a cell obtained from a diseased patient or alternatively a cell that has been treated to produce a cell having impaired function.

In one embodiment, the method further comprises a competition type assay, wherein, the method comprises contacting a competitive inhibitor compound with the pericyte cell either before or after the candidate compound is contacted with the cell. Preferably, the competition compound modulates the contractile state to decrease or increase the contractile state of the pericyte.

### Retina Screening Assay

The present invention provides methods for determining or identifying compounds that modulate the function of a blood vessel in an isolated retina, wherein a change in the contractile state of a blood vessel in an isolated retina is determined in the presence of a test compound, said change indicating that the test compound modulates blood vessel function. This embodiment of the invention does not encompass contacting an isolated blood vessel or capillary ex vivo with a test compound. In one embodiment, the invention provides a method for determining or identifying a compound that modulates the contractile state of a blood vessel in an isolated retina comprising contacting an isolated retina with a test compound and determining a change in the contractile state of said blood vessel, wherein said change indicates that the compound modulates the contractile state of the blood vessel.

In a preferred embodiment, the retina is a whole retina. In an alternate embodiment, the retina is not a whole retina. In one embodiment, the retina is a segment of a retina. In one embodiment, the test compound is administered in droplet form. In one embodiment, the test compound is administered as drops, vapour, atomised drops, nanoparticles, microspheres or mixtures thereof. In an alternate embodiment, the test compound is administered in a stream of solution. In one embodiment, the test compound is administered by perfusion.

In a further embodiment, the method comprises incubating the isolated retina in the presence of a test compound.

Preferably, the contractile state of the blood vessel is determined before the compound is contacted with the isolated retina, thereby facilitating a determination of the change in contractile state of the blood vessel. Alternatively, in certain preferred assay formats, the blood vessel generally assumes a contracted state in the absence of a test compound, in which case relaxation of the blood vessel or a reduced contractile state will be readily detectable.

Any art recognised means can be used to determine the contractile state of a blood vessel, such as, for example, visual detection, second messenger assay involving a determination of cAMP levels or Ca²⁺ efflux, determining blood vessel permeability, and the like.

The contractile state of a blood vessel is preferably detected by visual means, such as, for example, employing a microscope or similar analytical tool wherein the size, dimensions or volume of the blood vessel in vivo or in vitro or in situ and change thereto can be detected.

Preferably, the inventive method employs a technique for visualisation, isolation, treatment or maintenance of blood vessels such that their contraction or relaxation can be readily detected, and subjected to quantitation.

In another embodiment, the contractile state of a blood vessel is determined by a non-visual means.

The inventive method clearly encompasses the sequential contact of an isolated retina with a range of different compounds, that may be protagonistic or antagonistic towards each other. An isolated retina may be washed between contacts to remove the residual first compound or reduce the concentration of said compound to level that is too low to exert an effect on the contraction of a blood vessel.

In an alternative embodiment, the method further comprises contacting the isolated retina with a second test compound without washing the isolated retina between contacts with said first and second test compounds. In such cases, a second compound that is antagonistic towards the first compound will reverse the effects of the first compound if present at a sufficiently high concentration. Alternatively, where the second compound is a protagonist of the first compound, the contractile state of the blood vessel may be enhanced step wise (ie enhanced contraction or enhanced relaxation).

In one embodiment, the isolated retina is suspended freely in aqueous solution, the isolated retina is not in physical contact with a support.

In an alternate embodiment, the isolated retina is in physical contact with a support.

In a particularly preferred embodiment, the isolated retina is fixed to a support. The term “fixed” as used herein means directly or indirectly adhered to sufficiently stabilise the isolated retina. In one embodiment an adhesive agent is used to fix the isolated retina to the support. In a preferred embodiment, the adhesive agent is any of the following: cyanocrylate glue (“Super-glue”), 2 part resin (“Araldite”), high viscosity vacuum grease or silicone (“Sylgard”) or the like. In another embodiment an adhesive agent is not used. In one embodiment, the isolated is fixed to the support by virtue of a vacuum.

In a particularly preferred embodiment, the present invention provides a method of identifying a compound that modulates the contractile state of a blood vessel in an isolated retina comprising

providing an isolated retina fixed to a support;

contacting a test compound with said isolated retina; and

determining a distortion in said blood vessel, wherein said distortion indicates that said compound modulates the contractile state of a blood vessel in an isolated retina.

In one embodiment, the isolated retina is aligned to the interior surface of the posterior segment of an eye. The term “aligned” as used herein means substantially conformed to the shape of

Preferably, the posterior segment forms a cup that contains the retina in its normal physiological position. In one embodiment, the posterior segment is fixed to the support.

In an alternate embodiment, the isolated retina is not aligned to the interior surface of the posterior segment of an eye. In another embodiment, the isolated retina is aligned to a non-naturally occurring or synthetic surface. In one embodiment, the non-naturally occurring or synthetic surface is directly fixed to the support and the retina which is aligned thereto is thereby indirectly fixed to the support. In an alternate embodiment, the non-naturally occurring or synthetic surface is indirectly fixed to the support.

In one embodiment a segment of the non-naturally occurring or synthetic surface is substantially concave. Preferably, the substantially concave surface forms a cup that contains the retina in its normal physiological position.

In another embodiment, the non-naturally occurring or synthetic surface is not concave. In one embodiment, at least a segment of the surface is level or planar. By “level or planar” means that the surface is sufficiently flat such that surface does not maintain the retina in its normal physiological position. In another preferred embodiment the isolated retina is mounted flat onto a level or planar surface.

In one embodiment, the synthetic surface is constructed of glass.

In another embodiment, the synthetic surface is constructed of a non-glass material, such as a polymeric material. Mixed synthetic surfaces of glass and polymer are preferred.

Preferably the support is a surface, container or housing to which the isolated retina can be fixed. In one embodiment, the support is a container which is capable of containing solution. In one embodiment the support is a shallow dish, such as for example, a Petri dish. In another embodiment, the support is a micropipette.

Preferably, the test compound is contacted with the vitreous side of the isolated retina. Such contact is considered as a direct analogy for a non-invasive route of administration of a drug in a subject. In another embodiment the test compound is contacted with the non-vitreous side of the isolated retina. Non-vitreous contact of a test compound may be useful for determining and analysing drugs for invasive routes of administrating a drug in a subject.

The term “distort” and “distortion” as used herein refers to a change from the natural, previous, normal, or original shape or condition of a retinal blood vessel.

Preferably a distortion is determined by detecting a change of the physiology of the blood vessel. In one embodiment the contractile state of a retinal blood vessel is determined by measuring or otherwise determining a change in the size, dimensions or volume of the retinal blood vessel in vivo or in situ.

Preferably the distortion is visualised as a change of the thickness or diameter of a blood vessel in the isolated retina. In a preferred embodiment, the distortion is visualised as a change of the cross-sectional diameter of the blood vessel. As used herein the term “cross-sectional diameter” refers to the width of the blood vessel that is perpendicular to the long axis of the blood vessel.

In one embodiment an image of the retina is recorded. Images can be recorded using a digital camera or video camera. Preferably, the digital camera or video camera is attached to the optical viewing pathway of a microscope. In one embodiment, the cross-sectional diameter of the blood vessel is measured in pixels or in microns by using an appropriate calibration scale.

Preferably, the cross-section diameter can be visually measured by counting the number of pixels that comprise the cross-section diameter of the blood vessel in an image of the retina.

In one embodiment, the pixels corresponding to the cross-section diameter of the blood vessel can be identified by a change in contrast of the blood vessel compared to background retina. In one embodiment an image analysis tool is used to identify a change. Particularly specified image analysis tools include (i) a cross-section line profile of the pixel brightness made across the blood vessel so as to include the cross-section diameter and sections of the background retina, or (ii) a binary image of the retina that is used to create a distance map that quantifies the distance from each pixel in the image of the blood vessel to the nearest background pixel. Preferably, the sensitivity of measurement is 2 pixels.

Preferably, the images are not subjected to image enhancement prior to analysis.

In one embodiment, the blood vessel contains blood. Preferably the blood vessel contains at least a sufficient volume of blood which can be visibly detected (possibly with the use of a microscope), or otherwise detected. In another embodiment, the blood vessels contain a dye, such as for example Evans Blue dye. Preferably, the dye is perfused into the blood vessels. Preferably, the blood vessel contains an amount of the dye to enable detection visually or otherwise.

In one embodiment, the method further comprises:

(i) washing the isolated retina with a suitable buffer or aqueous solvent that is not damaging to the integrity of the retina or contractile function of the retinal blood vessels for a time and under conditions sufficient to remove the compound or reduce its activity to a level that does not affect blood vessel contraction;

(ii) optionally determining a distortion in the retinal blood vessel;

(iii) contacting the retinal blood vessel with a second test compound; and

(iv) determining a distortion in said retinal blood vessel, wherein said distortion indicates that said compound modulates the contractile state of the retinal blood vessel.

In an alternative embodiment, the method further comprises contacting the isolated retina with a second test compound without washing the isolated retina with a suitable buffer or aqueous solvent under conditions sufficient to remove the compound or reduce its activity to a level that does not affect retinal blood vessel contraction, wherein a further distortion in the retinal blood vessel indicates that the compound modulates the contractile state of the retinal blood vessel.

In either embodiment, if the second distortion is opposed to the distortion obtained for the first test compound, then the second compound is an antagonist of the first compound. If the second distortion amplifies the distortion achieved for the first test compound, then the second compound is a protagonist of the first compound. For example, a first test compound may induce relaxation of a retinal blood vessel, thereby increasing the cross-section diameter of the blood vessel, whereas a second compound may induce retinal blood vessel contraction, thereby decreasing the cross-sectional diameter of the retinal blood vessel. On the other hand, if both the first and second compounds produce the same effect, the diameter of the retinal blood vessel may be enhanced in a step wise manner between the addition of the first and second compounds (for protagonists of retinal blood vessel function) or reduced in a step wise manner (for antagonists of retinal blood vessel function). All such possibilities are encompassed by the invention.

Preferably, the method further comprises comparing a distortion of a retinal blood vessel from a subject with a control retinal blood vessel which can be treated (by contacting with a candidate compound) or untreated. In one embodiment, the control retinal blood vessel is a healthy retinal blood vessel. In an alternate embodiment, the control retinal blood vessel is an unhealthy retinal blood vessel such as for example a retinal blood vessel obtained from a diseased patient or alternatively a retinal blood vessel that has been treated to produce a retinal blood vessel having impaired function.

In one embodiment, the method further comprises a competition type assay, wherein, the method comprises contacting a competitive inhibitor compound with the retinal blood vessel either before or after the candidate compound is contacted with the retinal blood vessel. Preferably, the competition compound modulates the contractile state to decrease or increase the contractile state of the retinal blood vessel.

The retinal blood vessels and in particular the retinal capillaries have the highest ratio of pericyte to endothelial cell in any organ of the body. Accordingly, an agonist that will modulate the contractile state of a pericyte cell is expected to modulate the contractile state of a retinal blood vessel. A compound that will induce a contraction in a pericyte cell is expected to induce a contraction in a retinal capillary. Conversely, a compound that will induce an isolated pericyte to relax is expected to induce a retinal capillary to relax.

Those skilled in the art will be aware that whether or not contraction or dilation of a blood vessel occurs may, in some circumstances, be dependent upon the concentration of a compound applied to the pericyte or intact retina, as the case may be. The actual concentration of a compound used to elicit a contraction or dilation of a pericyte or a blood vessel in an isolated retina may also vary, such as, for example, as a consequence of variable uptake of the compound into the pericyte compared to the retina. Clearly, there may also be a difference in contractility or relaxation of the pericyte or blood vessel for any concentration of a compound over time (e.g., as the compound is utilized or metabolized). However, the concentration of any compound referred to herein that is required to elicit a contraction or dilation of a pericyte or a blood vessel in an isolated retina, or even in vivo, can be readily determined by the skilled artisan without undue experimentation.

Preferably, a compound referred to herein will elicit a relaxation of a pericyte in a pericyte assay, or dilation of a blood vessel in a retinal assay, or in vivo, at a concentration of the compound that is lower than the concentration of the compound required to elicit a contraction. For example, a concentration of a compound (e.g. an NSAID e.g., selected from the group consisting of aspirin and flurbiprofen, especially r-flurbiprofen) of more than about 3 micromolar may enhance contractility of a pericyte in the pericyte assay described herein, whereas a lower concentration of the compound may enhance relaxation (i.e. decrease contractility) of the pericyte. For the same compound, a concentration of less than about 10 micromolar when applied to the isolated retina in the retinal assay described herein will generally enhance dilation of the blood vessel in the retina. An initial dilation may be followed by a contraction or return to normal pericyte or blood vessel diameter over time, generally at about 5-10 minutes following administration of a compound, or longer.

### Diagnostic and Therapeutic Applications

The compounds identified in the novel screens of the invention are particularly useful in the diagnosis of impaired retinal blood vessel function and impaired pericyte function in a subject. Compounds that have been shown by in vitro screening to modulate retinal blood vessel function and pericyte function can be tested for safety and efficacy in animal models, such as for example, dogs, or rabbits, or mice, or guinea pigs, or rats or other small rodent, or pigs, and then proceed to clinical trials in humans, if desired. Naturally, for veterinary applications, no clinical trial in humans is required. Those compounds that are safe and efficacious in animals or humans can be applied to the eye of an appropriate subject to test for normal or healthy retina function. For subjects suffering from impaired retinal blood vessel function or pericyte function, such as, for example, glaucoma, corneal angiogenesis, retinopathy of prematurity, or diabetic retinopathy, agonist compounds will generally either fail to elicit a contraction when applied to the surface of the eye, or alternatively, elicit a slowed or incomplete contraction compared to the contractile state achieved for a healthy subject under similar or identical conditions.

A second aspect of the invention provides a method of diagnosing impaired retinal blood vessel function in a subject comprising administering to the subject an amount of a pharmaceutically acceptable compound that modulates blood vessel function under conditions sufficient to modify the contractile state of a blood vessel, wherein said compound is identified by a method comprising determining a change in the contractile state of a blood vessel in an isolated retina in the presence of said compound, wherein said change is indicative that the compound modulates blood vessel function, and detecting a change in the contractile state of the subject's retinal blood vessels. Preferably, the change in the contractile state of the subject's retinal blood vessel(s) is determined by a comparison with the change in contractile state of a retinal blood vessel of a healthy subject.

In a related aspect the invention provides a method of diagnosing impaired pericyte function in a subject comprising administering to the subject an amount of a pharmaceutically acceptable compound that modulates pericyte function under conditions sufficient to modify the contractile state of a pericyte and determining the change in the contractile state of the subject's pericytes, wherein said compound is identified by a process comprising determining a change in the contractile state of a pericyte in the presence of said compound, and wherein said change is indicative that the compound modulates pericyte function. Preferably, the change in the contractile state of the subject's pericytes is determined by a comparison with the change in contractile state of a pericyte of a healthy subject.

In one embodiment, the present invention provides a method of diagnosing retinal blood vessel damage in a subject, the method comprising:

(i) administering to the subject an amount of a pharmaceutically acceptable compound that modulates the contractile state of a pericyte wherein said compound is identified by a process comprising determining a change in the contractile state of a pericyte in the presence of said compound, wherein said change is indicative that the compound modulates pericyte function; and

(ii) detecting dilation or constriction of a capillary in the retina of the subject, wherein a slow or unsubstantial dilation or constriction of the retinal vessel indicates retinal vessel damage.

In another embodiment, the present invention provides a method of diagnosing retinal blood vessel damage in a subject, the method comprising:

(i) administering to the subject an amount of a pharmaceutically acceptable compound that modulates the contractile state of a blood vessel in an isolated retina wherein said compound is identified by a process comprising determining a change in the contractile state of a retinal vessel in an isolated retina in the presence of said compound, wherein said change is indicative that the compound modulates retinal vessel function; and

(ii) detecting dilation or constriction of a blood vessel in the retina of the subject, wherein a slow or unsubstantial dilation or constriction of the retinal vessel indicates retinal vessel damage.

In one embodiment, the retinal vessel is a capillary.

Preferably, the change in the contractile state of the subject's capillaries is determined by a comparison with the change in contractile state of a retinal capillary of a healthy subject.

Preferably, the method comprises contacting the subject's eye with an effective amount of the compound suitably formulated for veterinary or pharmaceutical use.

In one embodiment, the compound is administered using an invasive method. In one embodiment administration is by injection. In one embodiment administration is retrobulbar.

Particularly preferred routes of administration are non-invasive. In one embodiment, the route of administration includes iontophoretic administration. One preferred non-invasive route of administration of vasoactive compound includes use of iontophoresis applied to the cornea of an eye. In one embodiment the compound is administered in droplet form, such as, for example drops, vapour, atomised droplets, or by nanoparticles. Other forms of delivery particle, for example, microspheres and the like, are also contemplated for administration of the compound. Formulation of the compound or a liposome or other vesicle comprising said compound in suitable aqueous solvent for delivery by eye-dropper to the eye is particularly preferred.

Guidance in preparing suitable formulations and pharmaceutically effective vehicles, are found, for example, in Remington's Pharmaceutical Sciences, chapters 83-92, pages 1519-1714 (Mack Publishing Company 1990) (Remington's), which is hereby incorporated by reference. Further description of pharmaceutically effective vehicles are described further herein below.

The compounds identified in the screens of the invention are those small molecules, peptides, proteins, hormones, nucleic acids, organic or inorganic compounds etc that protagonise or antagonise contraction of a blood vessel in an isolated retina or a pericyte.

In one embodiment the compound is selected from the group consisting of: pituitary adenylate cyclase-activating polypeptide (PACAP), vasoactive intestinal polypeptide (VIP), a compound having activity on phospholipase C (PLC), a compound having activity on protein kinase A (PKA), a compound having activity on ion-channel hyperpolarisation channels, or a non-steroidal anti-inflammatory drug (NSAID).

Homologues, analogues or derivatives of the compound identified using the screens referred to herein are clearly contemplated herein, the only requirement being that such homologues, analogues and derivatives retain the same activity with respect to contractility function as the base compound from which they are derived, and preferably retain the ability of the base compound to modulate retinal blood vessel or pericyte contractile state.

In the case of proteinaceous compounds (ie peptides, polypeptides, enzymes and the like) functionally equivalent homologues are obtained from other sources, such as, for example viruses, bacteria, related organisms. Synthetic peptides, are particularly contemplated herein.

Particularly preferred modifications are those modifications designed to increase the stability of the identified peptides. Typical stabilising groups include amido, acetyl (eg at N-terminus), glycerol, benzoyl, phenyl, tosyl, alkoxycarbonyl, alkylcarbonyl, benzyloxycarbonyl and the like group modifications. Additional modifications include using an “L” amino acid in place of as “D” amino acid, cyclisation of the polypeptide, and amide rather than an amino or carboxy-terminal to inhibit exopeptidase activity.

Another approach to modification is to link the peptides or proteins to a variety of polymers, such as polyethylene glycol (PEG) and polypropylene glycol (PPG)—see for example U.S. Pat. Nos. 5,091,176, 5,214,131 and 5,264,209.

The proteinaceous compounds, such as, for example, PACAP or VIP, can be synthesised by any of the techniques that are known to one of ordinary skill in the art, for example, synthetic chemistry techniques (eg solid phase synthesis for solution synthesis) and/or recombinant DNA techniques. Synthetic chemistry techniques (eg solid phase synthesis) may be preferred for reasons of purity, freedom from an undesired side products and ease of product purification. Techniques for chemically synthesising peptides of the invention are reviewed by Borgia and Fields, 2000, TibTech 18:243-256 and described in detail in the references contained therein.

Alternatively, the proteinaceous compounds, such as, for example, PACAP or VIP, can be produced by recombinant DNA techniques in a host cell transformed with the nucleic acid having a sequence encoding such peptide. To produce a peptide by recombinant techniques, host cells (eg bacterial cells such as E. coli insect cells, yeast, or mammalian cells, for example, Chinese hamster ovary cells are transformed with a vector suitable for expressing a peptide of the invention and cultured in a medium such that the cells produce peptides). Peptides so-produced can be purified from cell culture medium, host cells, or both using techniques known in the art for purifying peptides including ultrafiltration, ion-exchange chromatography, gel filtration chromatography, electrophoresis or immunopurification with antibodies specific for the peptide.

Preferably, proteinaceous compounds are purified substantially free of conspecific proteins.

Non-steroidal anti-inflammatory drugs (NSAIDs) are a family of medications used to treat rheumatoid arthritis, osteoarthritis, mild-to-moderate pain, menstrual cramps, bursitis, gout, and migraine headaches, as well as other conditions. Ophthalmic formulations of certain NSAIDs are used during or after eye surgery. NSAIDs are divided into two categories, based on their action within the body: COX-1 and COX-2 inhibitors.

Examples of NSAIDs, are aspirin, pyrazalones, fenamate, diflunisal, acetic acid derivatives, propionic acid derivatives, oxicams, fenamates such as mefenamic acid, meclofenamate, phenylbutazone, diflunisal, diclofenac, Voltaren, indomethacin, sulindac, N-phenylanthranilic acid, etodolac, ketorolac, nabumetone, tolmetin, ibuprofen, fenoprofen, flurbiprofen, carprofen, ketoprofen, naproxen, piroxicam, indomethacin and flufenamic acid, or mimetics or derivatives thereof.

Guidance for alternate examples (including trade names) of NSAID's can be found for example, at the website of eMedicine (eMedicine Clinical Knowledge Base), San Francisco, Calif., USA, or alternatively, the website of Government Computer News in the headquarters for PostNewsweek Tech Media, Washington, D.C. 20002, USA.

Preferably, the non-steroidal anti-inflammatory drug is N-phenylanthranilic acid or flufenamic acid or flurbiprofen. More preferably, flurbiprofen in the R-isomer form, or S-isomer form.

Other compounds of interest include for example isoproterenol, dibutyryl cAMP forskolin, angiotensin II and endothelin-1.

The compound is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components of the pharmaceutical composition are adjusted according to routine skills in the art.

Optionally, the compound formulation will also include a carrier, such as, for example, to reduce surface denaturation of the compound if it is present at a low concentration in the formulation. Commonly used carrier molecules are bovine serum albumin (BSA), ovalbumin, mouse serum albumin, rabbit serum albumin and the like. Synthetic carriers also are used and are readily available. Carriers may be conjugated to the active compound. Means for conjugating polypeptides to carrier proteins are also well known in the art and include glutaraldehyde, m-maleimidobencoyl-N-hy-droxysuccinimide ester, carbodiimide and bis-biazotized benzidine.

It may also be desirable to co-administer biologic response modifiers (BRM) with the compound, to down regulate T cell responses or antibody responses to the compound.

Preferred vehicles for administration of the compound include liposomes. Liposomes are microscopic vesicles that consist of one or more lipid bilayers surrounding aqueous compartments. (Bakker-Woudenberg et al., Eur. J. Clin. Microbiol. Infect. Dis. 12 (Suppl. 1), S61 (1993); and Kim, Drugs 46, 618 (1993)). Liposomes are similar in composition to cellular membranes and as a result, liposomes generally are administered safely and are biodegradable.

Techniques for preparation of liposomes and the formulation (e.g., encapsulation) of various molecules, including peptides and oligonucleotides, with liposomes are well known to the skilled artisan.

Depending on the method of preparation, liposomes may be unilamellar or multilamellar, and can vary in size with diameters ranging from 0.02 .mu.m to greater than 10 .mu.m. A variety of agents are encapsulated in liposomes. Hydrophobic agents partition in the bilayers and hydrophilic agents partition within the inner aqueous space(s) (Machy et al., Liposomes in Cell Biology and Pharmacology (John Libbey 1987), and Ostro et al., American J. Hosp. Pharm. 46, 1576 (1989)).

Liposomes can also adsorb to virtually any type of cell and then release the ncapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446, 368 (1985)). Irrespective of the mechanism or delivery, however, the result is the intracellular disposition of the associated compound.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as compounds.

Cationic liposome preparations are made by conventional methodologies (Feigner et al, Proc. Nat'l Acad. Sci USA 84, 7413 (1987); Schreier, Liposome Res. 2, 145 (1992)). Commercial preparations, such as Lipofectin (Life Technologies, Inc., Gaithersburg, Md. USA), are readily available. The amount of liposomes to be administered are optimized based on a dose response curve. Feigner et al., supra.

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MUV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. (See Colloidal Drug Delivery Systems, vol. 66, J. Kreuter, ed., Marcel Dekker, Inc. 1994).

Preferably, detecting a change in the contractile state of the subject's retinal blood vessels comprises measuring or otherwise detecting the change in size of volume of the retinal blood vessel.

In one embodiment, detecting a change in the contractile state of the subject's retinal blood vessels comprises using a microscope, ophthalmoscope or Fundus camera. In another embodiment detecting a change in the contractile state of the subject's retinal blood vessels is performed without using microscope, ophthalmoscope or Fundus camera. In one embodiment images of the retina are recorded. In one embodiment a camera, more preferably a digital camera is used to record the images.

Preferably the distortion is visualised as a change of the thickness or diameter of a blood vessel of the retina. In a preferred embodiment, the distortion is visualised as a change of the cross-sectional diameter of the blood vessel. As used herein the term “cross-sectional diameter” refers to the width of the blood vessel that is perpendicular to the long axis of the blood vessel.

In one embodiment images of the retina are recorded using a digital camera. Preferably, the digital camera is attached to the optical viewing pathway of a microscope. In one embodiment, the cross-sectional diameter of the blood vessel is measured in pixels or in microns by using an appropriate calibration scale.

Preferably, the cross-section diameter can be measured by counting the number of pixels that comprise the cross-section diameter of the blood vessel in an image of the retina.

In one embodiment, the pixels corresponding to the cross-section diameter of the blood vessel can be identified by a change in contrast of the blood vessel compared to background retina. In one embodiment an image analysis tool is used to identify a change. Particularly specified image analysis tools include (i) a cross-section line profile of the pixel brightness made across the blood vessel so as to include the cross-section diameter and sections of the background retina, or (ii) a binary image of the retina that is used to create a distance map that quantifies the distance from each pixel in the image of the blood vessel to the nearest background pixel. Preferably, the sensitivity of measurement is 2 pixels.

Preferably, the images are not subjected to image enhancement prior to analysis.

The subject may have one or more symptoms associated with diabetes mellitus, such as, for example, glaucoma, corneal angiogenesis, or diabetic retinopathy. Alternatively, the subject may have no visible symptoms developed in respect of diabetes mellitus or is otherwise considered healthy. Preferred healthy subjects will have normal blood glucose concentrations for their age, ethnicity, sex, and weight, preferably about 5 millimole/L.

It is preferred to administer to the subject being tested (healthy or non-healthy) an amount of the compound that is sufficient to induce a dilation of the retinal blood vessels including capillary blood vessels of a healthy subject. Such dilation is a consequence of relaxation of the cells including pericytes, which relaxation enhances the cross-sectional diameter of the retinal capillary vessels. Preferably, the increase in capillary diameter also leads to an alteration in the velocity of blood flow in the capillaries.

In the case of a subject having elevated blood glucose levels compared to a healthy subject, or alternatively, a subject suffering from early stage or advanced diabetes mellitus, the compound of the invention induces a reduced dilation of the retinal capillary blood vessels compared to the dilation observed for a healthy subject. Thus, the degree of pericyte relaxation induced for a diseased subject, or a subject having a propensity to develop glaucoma, corneal angiogenesis, or retinopathy is much reduced compared to a normal subject.

The diagnostic application of the present invention may also be useful in monitoring the progression of disease and/or treatment of disease in a subject in need thereof. The diagnostic application may be useful in determining the condition of a diseased subject or change thereof or the improvement or lack of improvement in a diseased subject. For example, the diagnostic application may be useful in determining the condition of a subject being treated to improve their diabetic condition and treated to reduce glucose levels in their blood. It is expected that as the patient's condition improves that the response of the retinal vessels to the vasoactive compound will normalise, ie., the response will be more similar to a healthy subject.

The present invention clearly contemplated a comparison between the subject being tested and a standardised or normalised response for a healthy subject, thereby obviating the need for a direct side-by-side comparison to reach a diagnosis. To determine a standardised or normalised response for a healthy subject, the time and extent of dilation of the retinal capillary are determined for a particular compound administered to a panel of healthy subjects having no familial history or symptoms of diabetes mellitus, glaucoma, corneal angiogenesis, retinopathy of prematurity, diabetic retinopathy or ocular disease and the mean responses of the panel are determined. Such epidemiological data are particularly useful for comparison to the response of a test subject to a particular compound being used in the diagnostic assay.

The foregoing embodiments apply mutatis mutandis to assays conducted in vivo using compounds that induce constriction of the retinal capillaries.

Preferably, the dilation response of the retinal capillary blood vessels in response to the compound has a threshold of sensitivity for subjects having blood glucose concentrations up to about 7.5 millimole/L, which includes at the higher end of the scale those subject having diabetic retinopathy. Accordingly, the present assay is particularly useful for diagnosing subjects in the early stages of developing diabetic retinopathy.

The compounds identified using the methods described herein are also useful for the therapeutic or prophylactic treatment of diseases associated with aberrant retinal vessel function, such as, for example, aberrant retinal vessel growth or contractility, retinopathy and other disorders or the eye associated with impaired retinal vessel function.

The compounds identified using the methods described herein are also useful for the therapeutic or prophylactic treatment of diseases associated with aberrant pericyte function, such as, for example, aberrant cell growth or contractility, retinopathy and other disorders or the eye associated with impaired pericyte function.

Accordingly, in a third aspect, the present invention provides for the use of a compound that modulates retinal blood vessel function in the preparation of a medicament for the treatment of impaired retinal blood vessel function in a subject, said compound being identified by a method comprising determining or identifying a change in the contractile state of a blood vessel in an isolated retina in the presence of said compound, wherein said change is indicative that the compound modulates retinal blood vessel function.

In a related aspect, the present invention provides for the use of a compound that modulates pericyte function in the preparation of a medicament for the treatment of impaired pericyte function in a subject, said compound being identified by a method comprising determining a change in the contractile state of a pericyte in the presence of said compound, wherein said change is indicative that the compound modulates pericyte function.

In other related aspects the invention provides methods for treating a subject having impaired pericyte function comprising administering to the subject an amount of a pharmaceutical composition comprising a compound that modulates pericyte function and a pharmaceutically acceptable carrier, diluent or excipient, wherein said compound is identified by a process comprising determining a change in the contractile state of a pericyte in the presence of said compound, wherein said change is indicative that the compound modulates pericyte function.

Preferably, the compound is administered under conditions sufficient to alleviate one or more impaired pericyte functions, including impaired pericyte contraction. The alleviation may be temporary, in which case repeated or continuous dosage of the compound may be required. Those skilled in the art will readily be in a position to determine an effective dosage regimen for the active ingredients identified in the inventive screens described herein.

For therapeutic applications (or in vivo diagnostic applications for that matter), it is preferred that the compound is administered to the subject's eye, more preferably the retina, in an amount sufficient to modulate pericyte contractile state of the pericyte. Such an amount can be determined empirically, preferably using animal models. The effective amount of the compound will vary according to factors such as the type of disease of the individual, the age, sex, and weight of, and the extent of retinal disease of the individual. Administered concentrations can be adjusted to provide the optimum diagnostic response. For example, several divided doses can be administered separately. Alternatively, combinations of compounds can have synergistic effects and the concentrations can be adjusted accordingly.

The forgoing embodiments apply mutatis mutandis to therapeutic applications to treat a subject having impaired retinal vessel function, or impaired pericyte function.

Preferably, the inventive method further comprises formulating a compound or a salt thereof identified by the methods of the invention in a suitable pharmaceutically acceptable carrier or diluent or excipient, and more preferably administering the compound to a subject in need- of treatment, such as, for example, a subject having impaired retinal blood vessel function or pericyte function or suspected of having impaired retinal blood vessel function or pericyte function.

Accordingly in another aspect, the present invention provides for the use of a compound that modulates retinal blood vessel function in the preparation of a medicament for the treatment of impaired retinal blood vessel function in a subject, said compound being identified by a method comprising determining or identifying a change in the contractile state of a blood vessel in an isolated retina in the presence of said compound, wherein said change is indicative that the compound modulates retinal blood vessel function.

In a related aspect, the invention provides for the use of a compound that modulates pericyte function in the preparation of a medicament for the treatment of impaired pericyte function in a subject, said compound being identified by a process comprising determining a change in the contractile state of a pericyte in the presence of said compound, wherein said change is indicative that the compound modulates pericyte function.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present invention is further described with reference to the following non-limiting examples

### EXAMPLE 1

### Preparation of Silicone Rubber Substrate

A small volume of dimethylpolysiloxane (Sigma Chemical Co.) of either 60,000 cps (DMPS-60M) or 12,500 cps (DMPS-12M) viscosity was applied to 12 mm diameter glass cover slips. In some cases an intermediate viscosity dimethylpolysiloxane (30,000 cps) was prepared by blending 54% by weight of 60,000 cps dimethylpolysiloxane with 46% by weight of 12,500 cps dimethylpolysiloxane. The coated cover-slip was heated for 2 seconds using a Bunsen burner to induce cross-linking of the surface of the dimethylpolysiloxane and formation of a thin silicone rubber sheet bonded to the cover slip. After preparation, the cover slips were placed in 24-well tissue culture dishes and sterilised by UV irradiation overnight.

### EXAMPLE 2

### Growth of Pericytes on Silicone Rubber Substrate

Pericytes from primary cultures (first passage) were plated on the cover slips in DMEM supplemented with 10% FCS. The experiments were performed 48 h later, when almost all the cells were spontaneously in a contracted state as manifest by wrinkles in the rubber sheet beneath the cells (FIG. 1).

All experiments were performed using only first passage cultures of pericytes to minimise any change in pericyte physiology that might occur in prolonged culture.

### EXAMPLE 3

### Evaluating the Contractility of Pericytes

The response of the cells to the antagonists was evaluated using phase contrast microscopy at room temperature. The spontaneous contractile tone of pericytes induced tension wrinkles that could be observed after 24 h (FIG. 1). Wrinkles were only measured when associated with a pericyte, as shown in FIG. 1. Cells were identified as relaxed when the tension wrinkles associated with them diminished in size, and completely relaxed when the wrinkles disappeared. Conversely, a pericyte contracted when there was an increase in the number and length of the wrinkles associated with the pericyte. During each experiment, an image of the pericytes was captured (videocamera and framegrabber) by the computer every minute. The wrinkles were analysed after the experiment from these captured images.

In each of the experiments, the length of each clearly discerned wrinkle associated with the cell was measured 3 times, and the average of the lengths was tabulated. The two ends of a wrinkle were determined by the contrast difference from the background on the picture. The contrast settings on the computer, videocamera or framegrabber were set at the commencement of an experiment and then not changed during the course of an experiment. Similarly, the focus of the microscope was set at the commencement of the experiment and then not changed during the course of an experiment. The depth (level) of solution in the bath was also kept constant, by aspirating and injecting each new solution at the same rate. Thus the only change in the image of the wrinkle (contrast, length) was due to the tension induced in the silicone substrate by the pericyte. The wrinkles during each experimental condition were counted from the photographs with help of a Zeiss videoplan.

The contractile state of the pericyte was quantified by counting the number of wrinkles (N) and the length of each wrinkle (l) in the thin shin sheet of silicone. From these observations an index of contractility (Cᵢ) was developed from N.times.1. An experiment to determine the effect of vasoactive agents was conducted by first quantifying Cᵢ with the pericyte in physiological buffer (“control”). The Cᵢ was quantified after exposing the pericytes to a vasoactive agent and allowing the pericyte to reach a steady-state level of contractility, which usually took 10 minutes. The effect of the vasoactive agent was determined by dividing by the Cᵢ in the control condition and multiplying by 100 to obtain an index of effect (Iₑ,%) that was either less than 100 (pericyte relaxed relative to control) or greater than 100 (pericyte contracted relative to control). As a further control, incubations with buffer alone for the complete duration of exposure to each vasoactive agent were performed in conjunction with each experiment.

### EXAMPLE 4

### Testing the Functionality of the Single-Cell Contractility Assay

Vasoactive agents might modulate the contractile activity of the cells adhering on the silicone substrate. It was confirmed that retinal capillary pericytes are contractile cells, and that the silicone substrate system could provide quantification of the contractility, by observing that norepinephrine, one of the most potent biogenic vasoconstrictors, caused a contractile response.

For this purpose cells were grown on silicone rubber. On the day of experiment the cells were rinsed with HEPES-buffered solution for 20 min at room temperature. Then the pericytes were exposed to norepinephrine (N5785, Sigma, USA) at different concentrations (10⁻⁶M, 10⁻⁵M, 10⁻⁴ M) by a rapid exchange of the entire solution. The pericytes were exposed to each new drug concentration for a total of 10 minutes, with an image of the pericytes taken every 1 min. Changes in the number of wrinkles associated with the pericytes were analysed from the images recorded every 1 min.

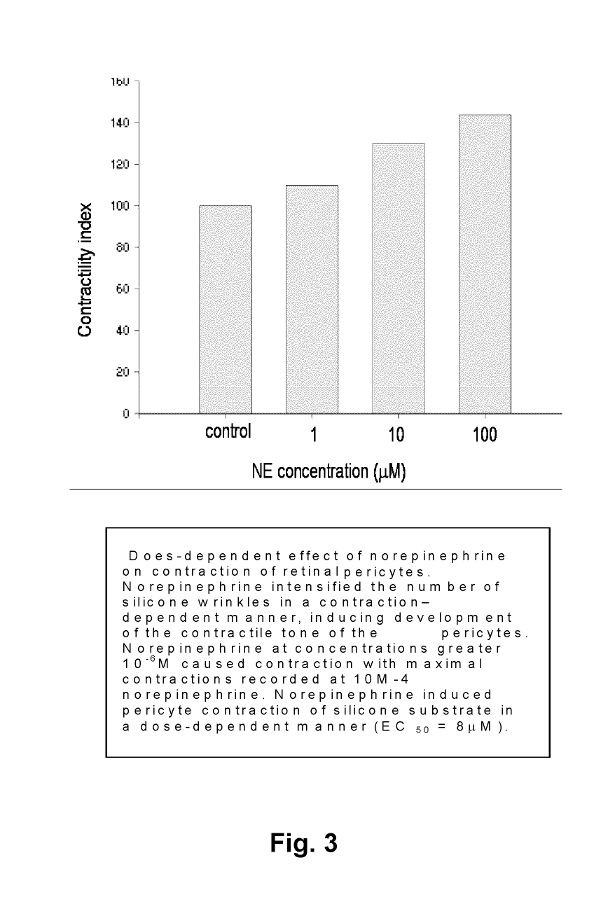

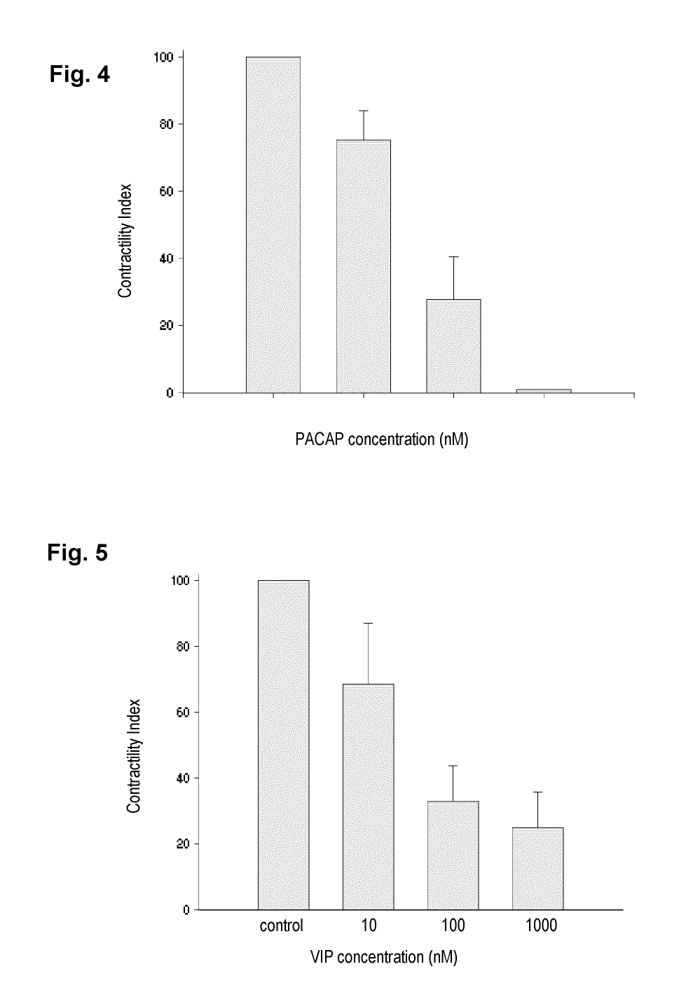

After calculating the index of effect, the experiment demonstrated that the contractility assay was capable of measuring changes in pericyte contractility and that pericytes were capable of contracting in a dose-dependent manner to norepinephrine (FIG. 3).

### EXAMPLE 5

### Effect of Vasoactive Peptides on Pericyte Contractility

The inventors have identified several potential vasoactive agents that can affect retinal capillaries. The vasoactive peptides subject to the present experiments are VIP (vasoactive intestinal polypeptide) and PACAP (pituitary adenylate cyclase activating polypeptide). The inventors have identified that these peptides can influence the contractile state of retinal pericytes, an effect that will influence capillary haemodynamics.

Pituitary adenylate cyclase-activating polypeptide (PACAP) is a hypothalamic peptide, having a potent action in stimulating cyclic adenosine 3′-monophosphate (cAMP) production in anterior pituitary cells. PACAP and VIP receptors are widely distributed, occurring in the central nervous system and peripheral organs, such as the eye.

The present inventors explored how PACAP and VIP affect capillary tone and thus play a role in vascular regulation through effect of pericyte contraction and relaxation. Experiments determined that pericytes are capable of responding to PACAP and VIP stimulation and in vivo can modulate microvessel lumina diameter and thus regulate local blood flow.

For this purpose cells were grown on silicone substrate. On the day of the experiment the cells were rinsed with HEPES-buffed physiological solution, and left in this solution for 20 min at room temperature. PACAP or VIP was added in increasing concentrations by fluid exchange over pericytes grown on silicone substrates. Pericytes were exposed for 10 minutes to each new concentration of PACAP or VIP. The effect of PACAP on pericytes was investigated and compared to that of VIP. The concentrations of PACAP were 10⁻⁹M, 10⁻⁸M, and 10⁻⁷M. The concentrations of VIP were 10⁻⁹M, 10⁻⁸M, and 10⁻⁷M.

The time course of the effect of a single dose of PACAP was studied when PACAP 10⁻⁸ M was applied for 20 minutes. After 20 minutes the solution containing the drug was removed, and the cells were washed with drug-free buffer. VIP and PACAP were purchased from AUSPEP (Australia).

The inventors identified that in one embodiment an increase in cAMP following the binding of PACAP or VIP could result in an increase in cAMP-dependent protein kinase activity. Cyclic 3′5′-adenosine monophosphate (cAMP) is an important intracellular second messenger in many tissues and mediates the effect of multiple drugs and hormones. cAMP regulates a number of different cellular processes such as cell growth and differentiation, ion channel conductivity, synaptic release of neurotransmitters, and gene transcription. Reversible protein phosphorylation is a key regulatory mechanism in eukaryotic cells.

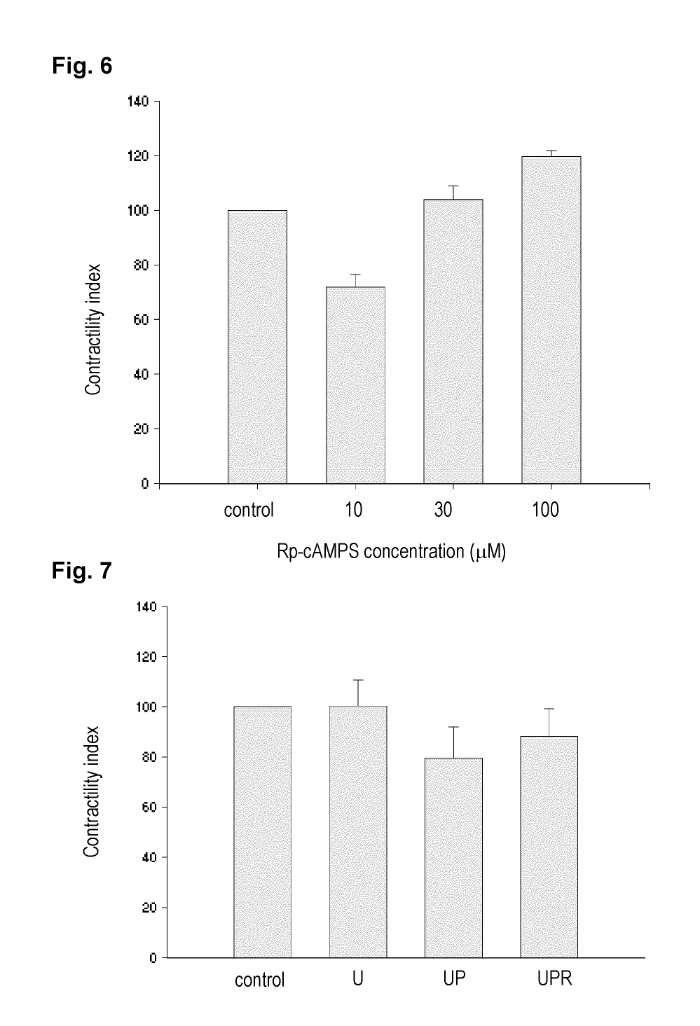

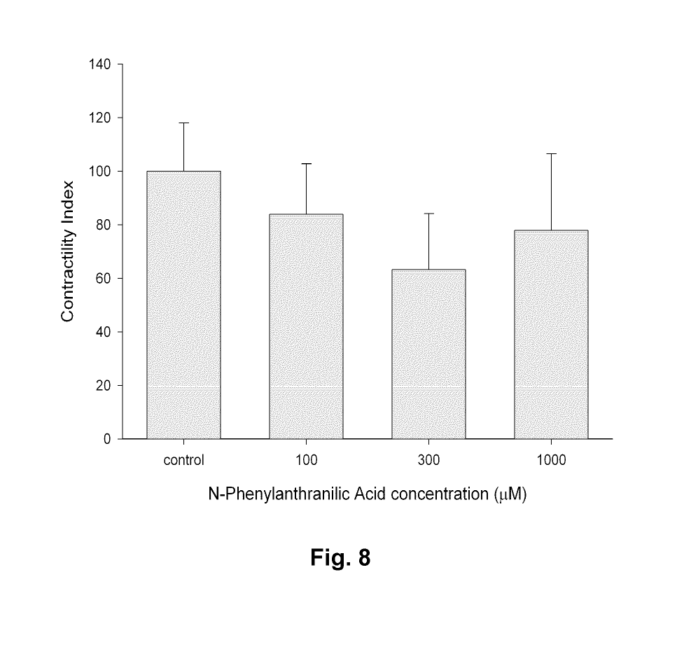

The present inventors further identified protein kinase A (PKA) as being affected by the increase in cAMP following the binding of PACAP or VIP. For this purpose cells were grown on silicone substrate and contacted by a 10⁻⁸ M concentration of PACAP. The drug adenosine 3′,5′-cyclic monophosphothioate, Rp-isomer (Rp-cAMPS, A7850 Sigma USA) is a specific inhibitor of PKA activity and was added sequentially into the PACAP 10⁸M solution in three separate concentrations (10 μM, 30 μM, and 100 μMcommencing with the lowest concentration. The pericytes were allowed to reach a steady-state in the altered contractile response (10 minutes) with each Rp-cAMPS concentration before changing the superfusing solution for the next concentration. We discovered that Rp-cAMPS, in a dose-dependent manner and with an EC₅₀ value of 26 μM, inhibited the relaxing effect of 10⁻⁸M PACAP. It was shown that the drug N-(2-[p-bromocinnamylamino]-ethyl)-5-isoquinolinesulfonamid-e (H-89, B 1427 Sigma USA) at a concentration of 0.3 μM, which is another specific inhibitor of PKA activity, inhibited the relaxing effect of 10⁻⁸ M PACAP. See FIG. 6.

The present inventors have also shown that the relaxing effect of PACAP on pericyte contraction was in part mediated by the intracellular pathway that mobilises Ca²+ from intracellular stores. The drug 1-[6-([(17.beta.)-3-Methoxyestra-1,3,5(10)-trien-17-yl]amino)hexyl]-1H-py-rrole-2,2-dione (U73122, U6756 Sigma USA) at a concentration of 10 μM, is a specific antagonist of intracellular pathways linked to phospholipase C (PLC), and is an antagonist of the PACAP-induced relaxation of pericytes.

### EXAMPLE 6

### Effect of Non-Steroidal Anti-Inflammatory Drugs on Pericyte Contractility

The inventors have identified several potential non-steroidal anti-inflammatory drugs (NSAID) that can affect retinal capillaries. The NSAID subject to the present experiments are N-phenylanthranilic acid, flufenamic acid and flurbiprofen. The R-isomer form of flurbiprofen was used in these experiments, and is referred to as R-flurbiprofen. The inventors have identified that these NSAID can influence the contractile state of retinal pericytes, an effect that will influence capillary haemodynamics.

The present inventors explored how N-phenylanthranilic acid, flufenamic acid and R-flurbiprofen affect capillary tone and thus play a role in vascular regulation through effect of pericyte contraction and relaxation. Experiments determined that pericytes are capable of responding to N-phenylanthranilic acid, flufenamic acid and R-flurbiprofen stimulation and in vivo can modulate microvessel lumina diameter and thus regulate local blood flow.

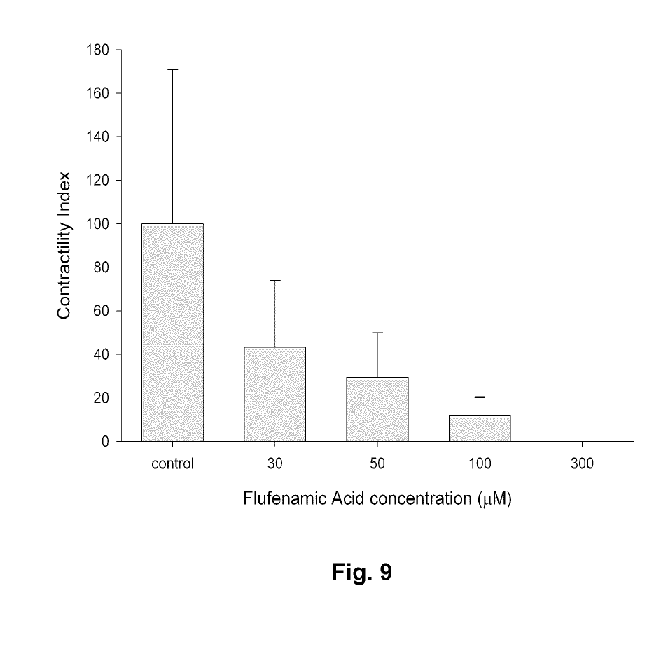

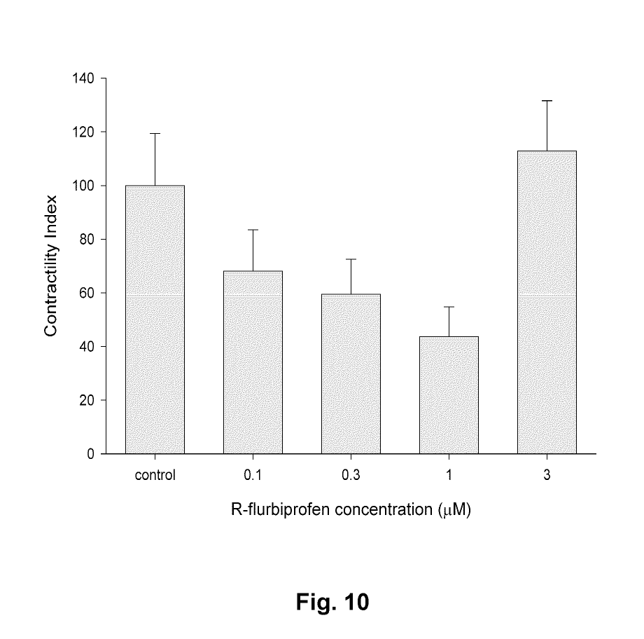

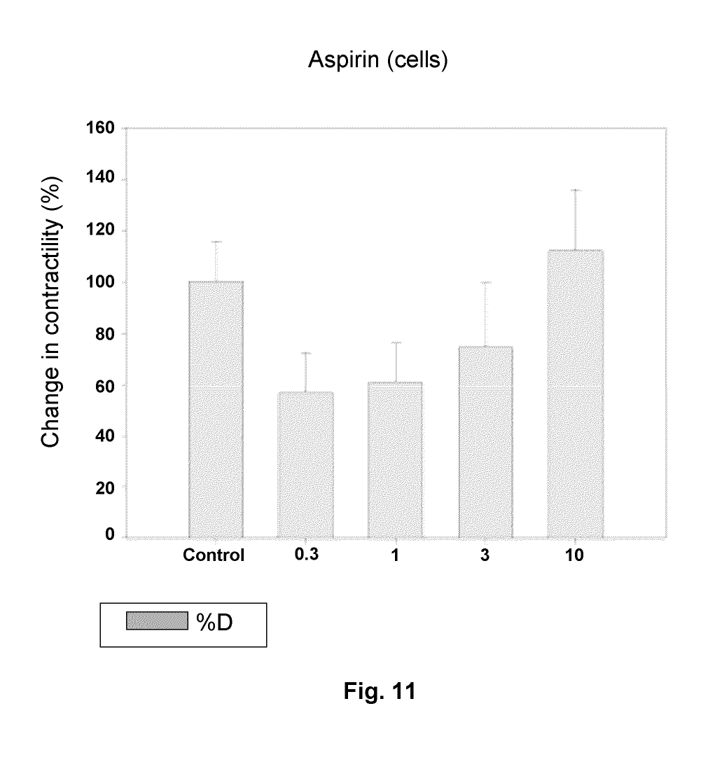

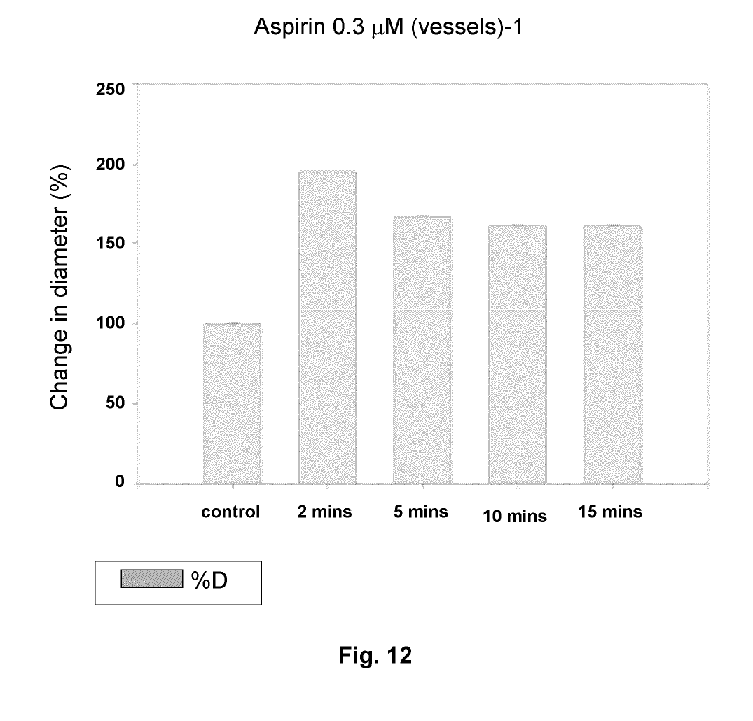

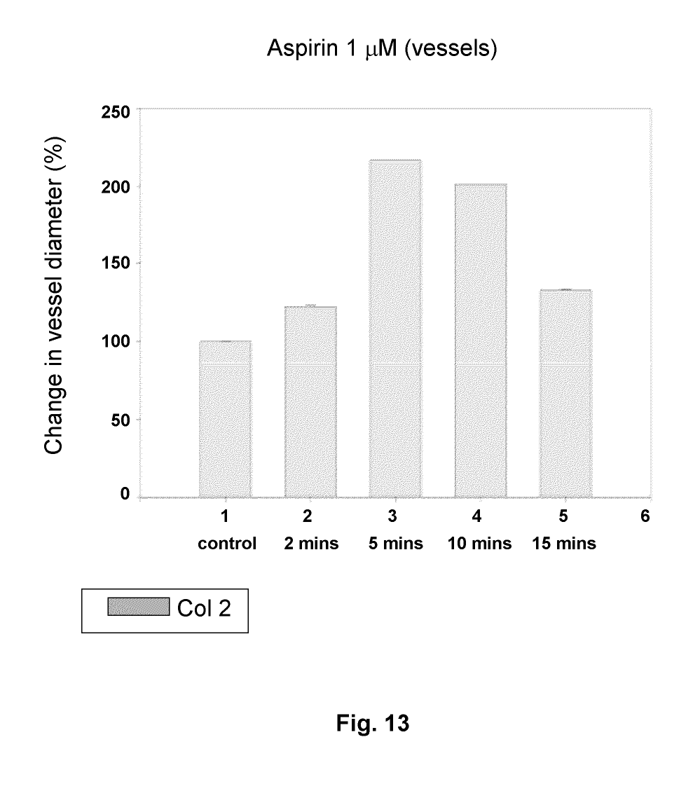

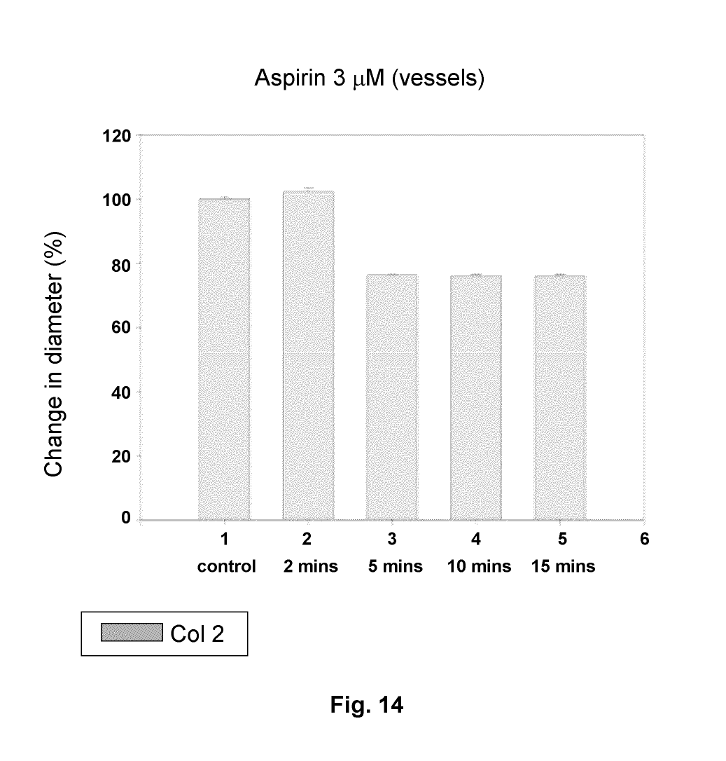

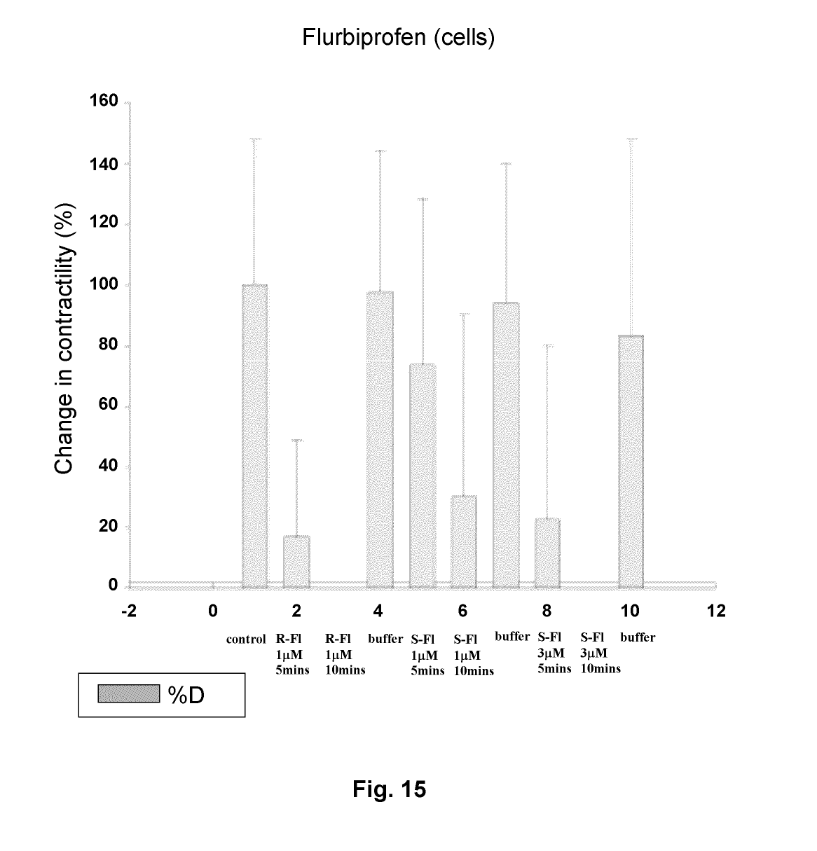

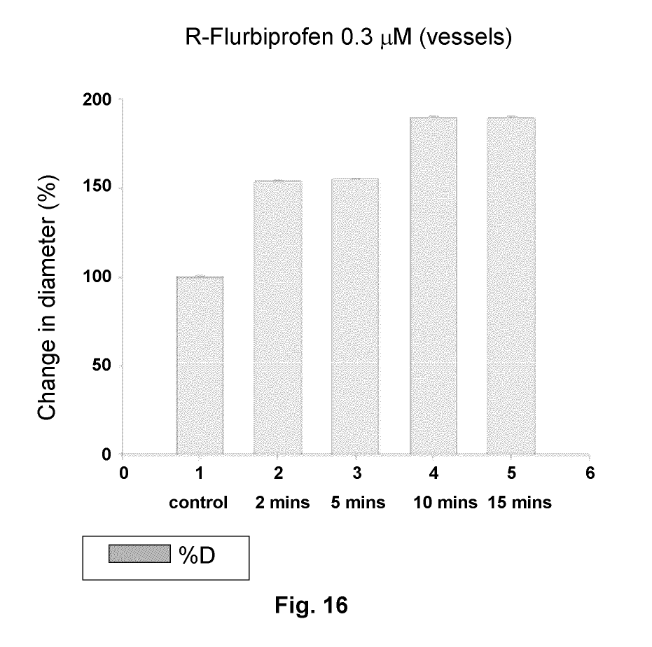

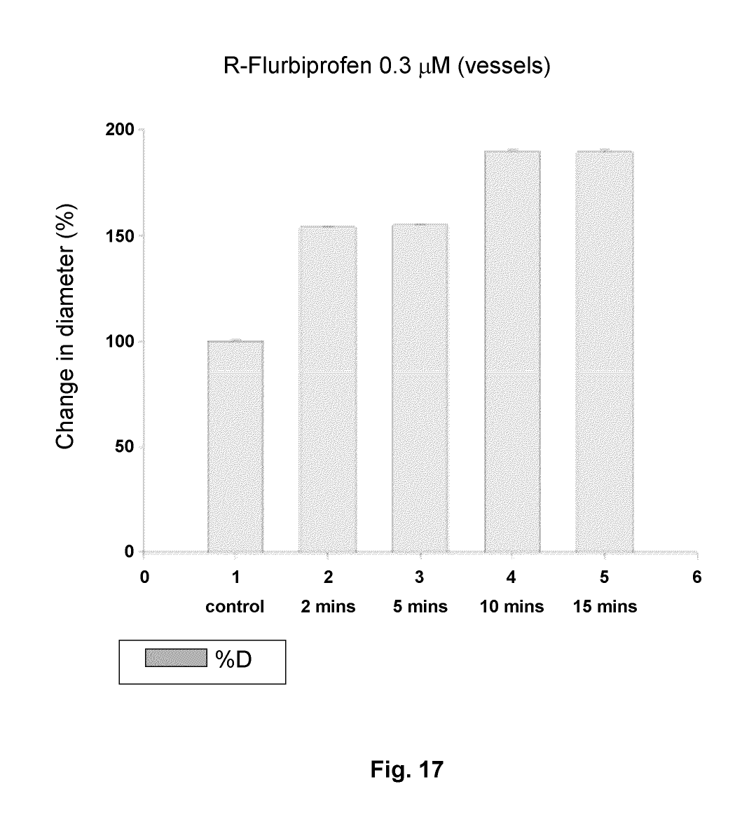

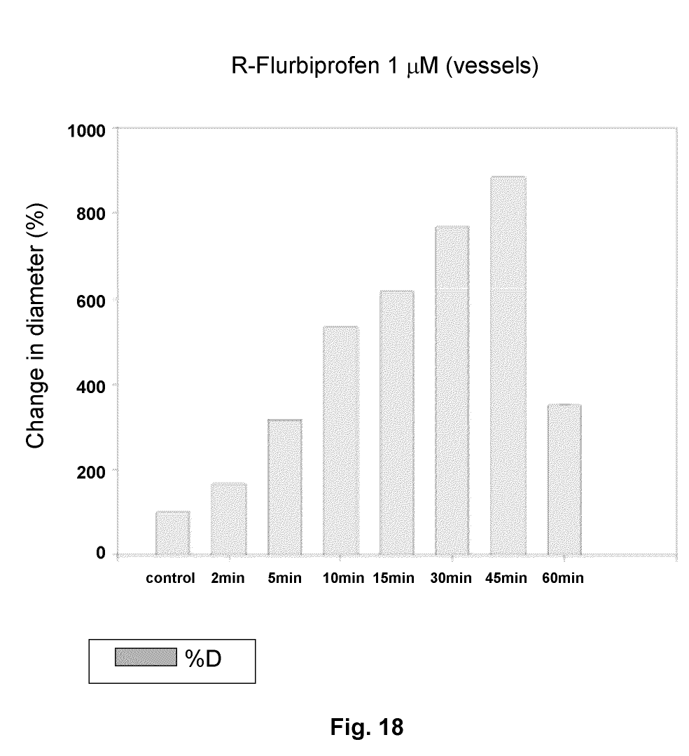

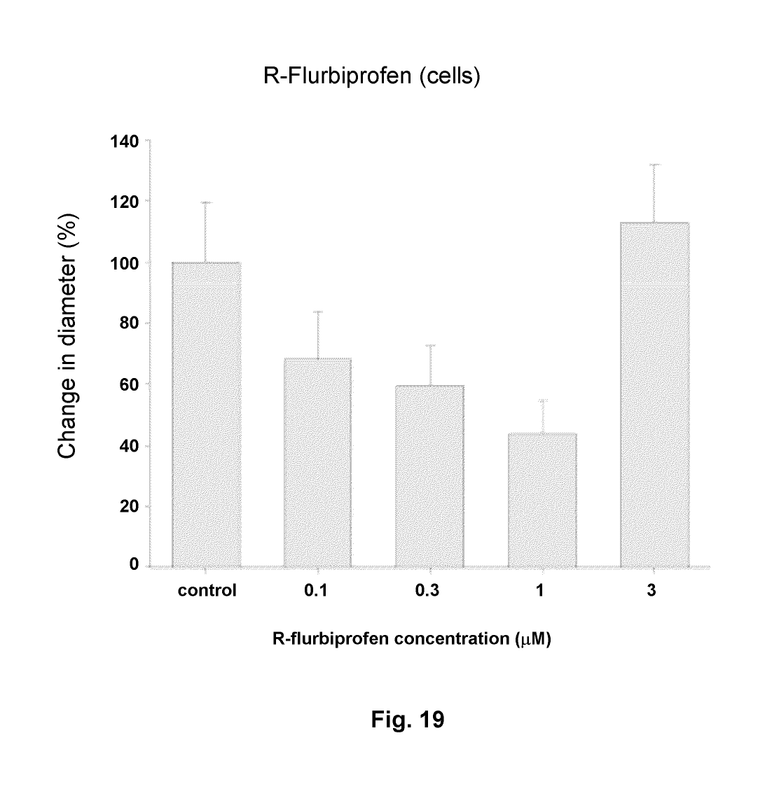

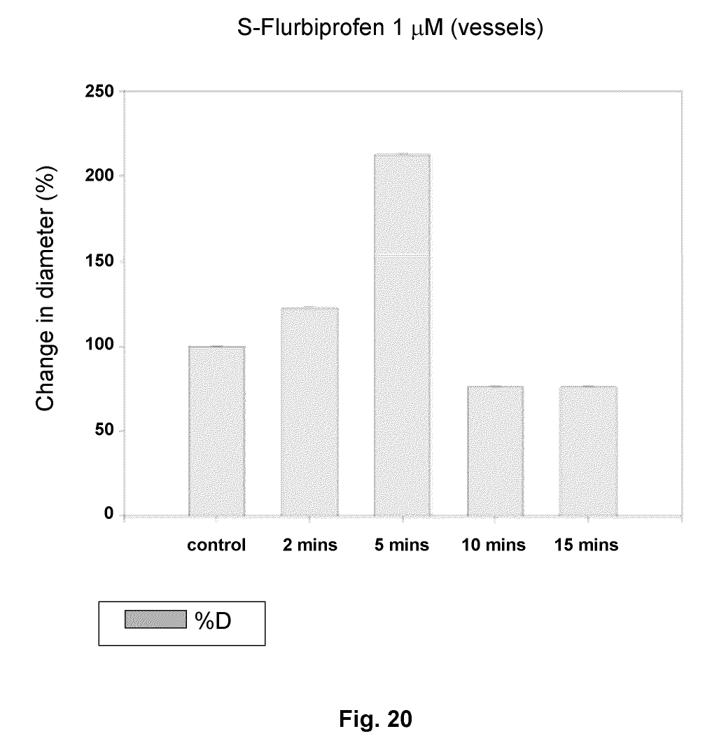

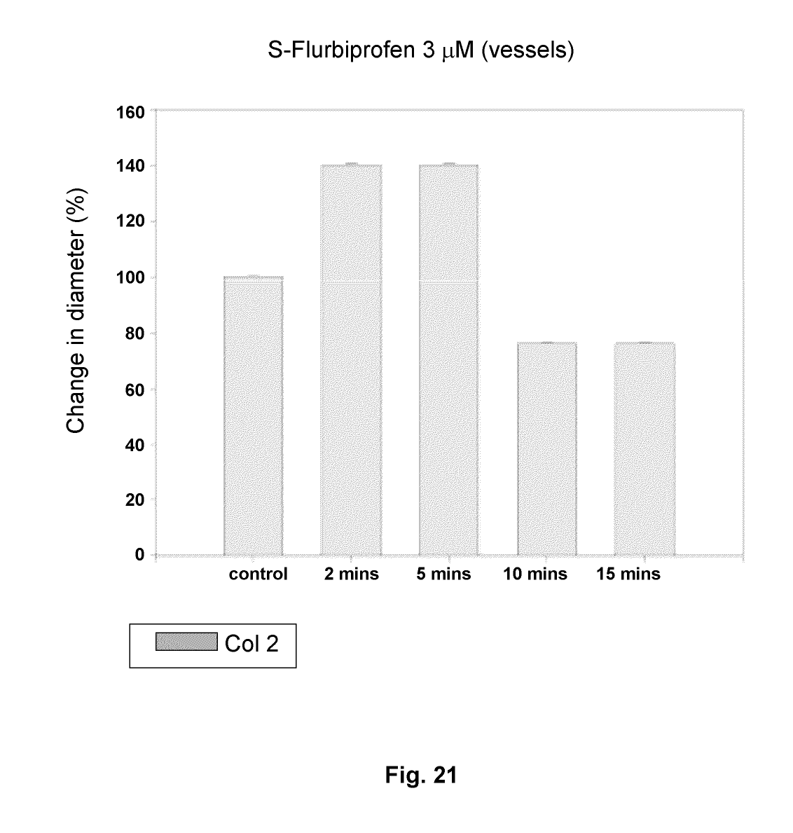

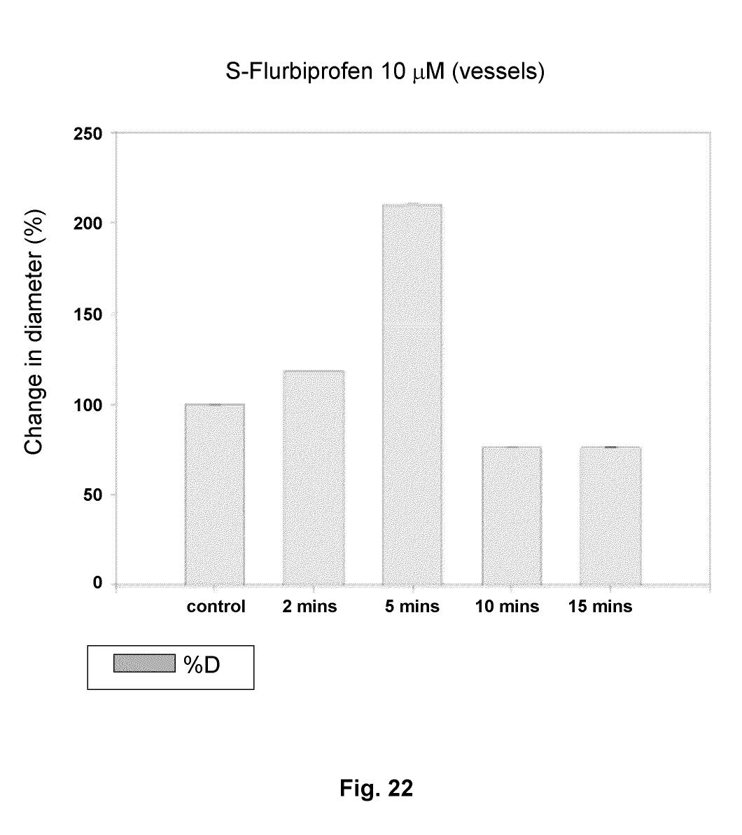

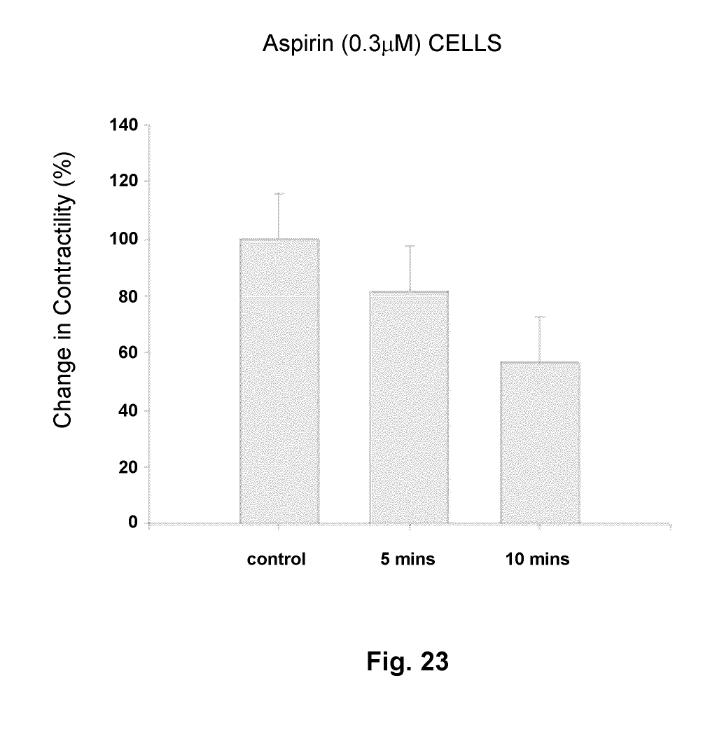

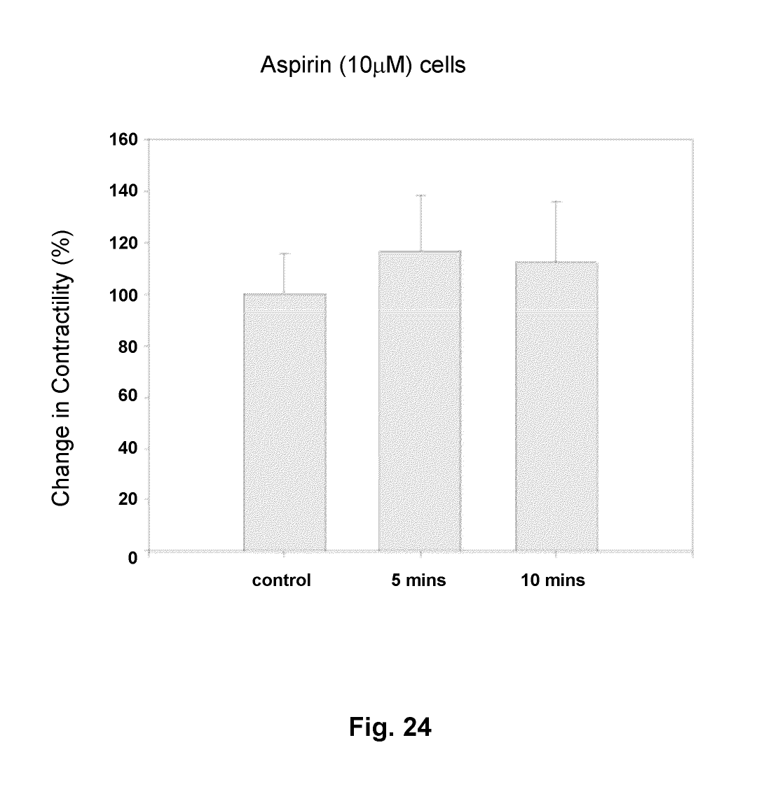

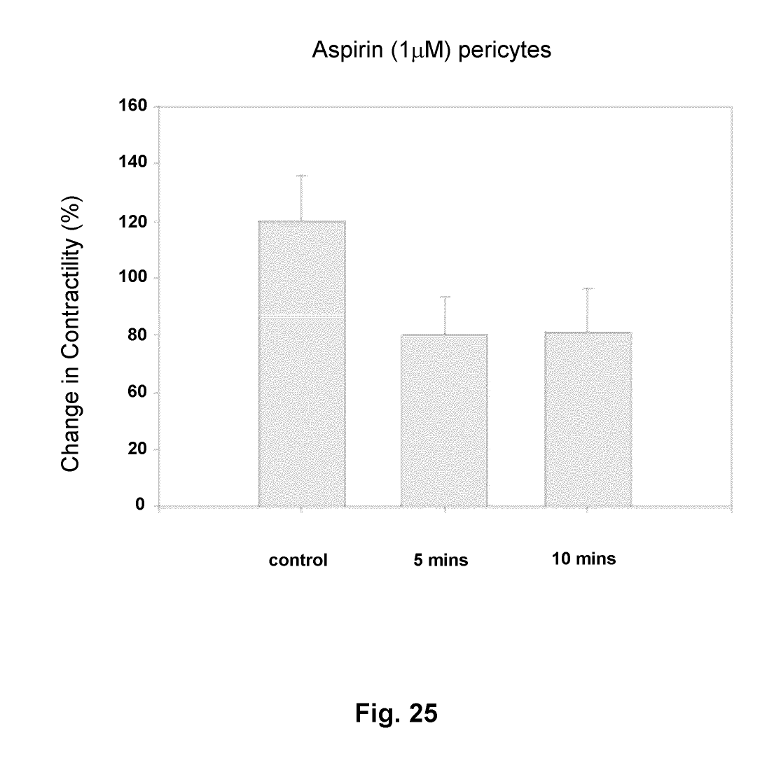

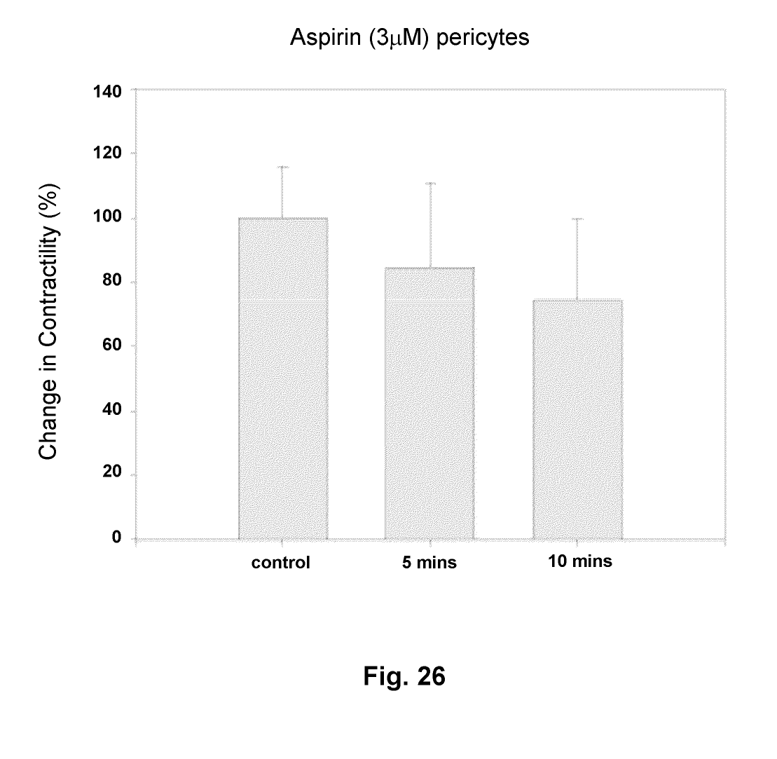

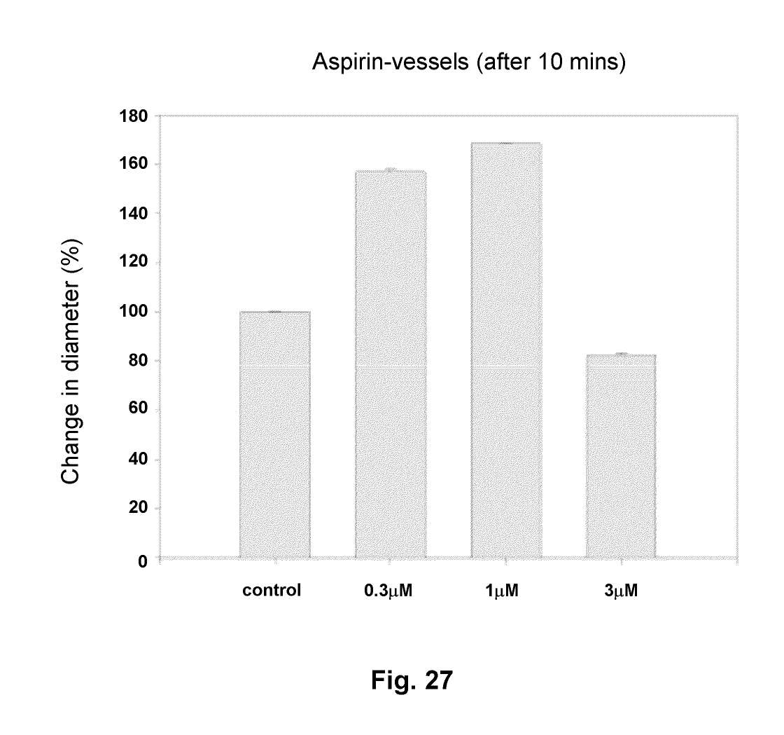

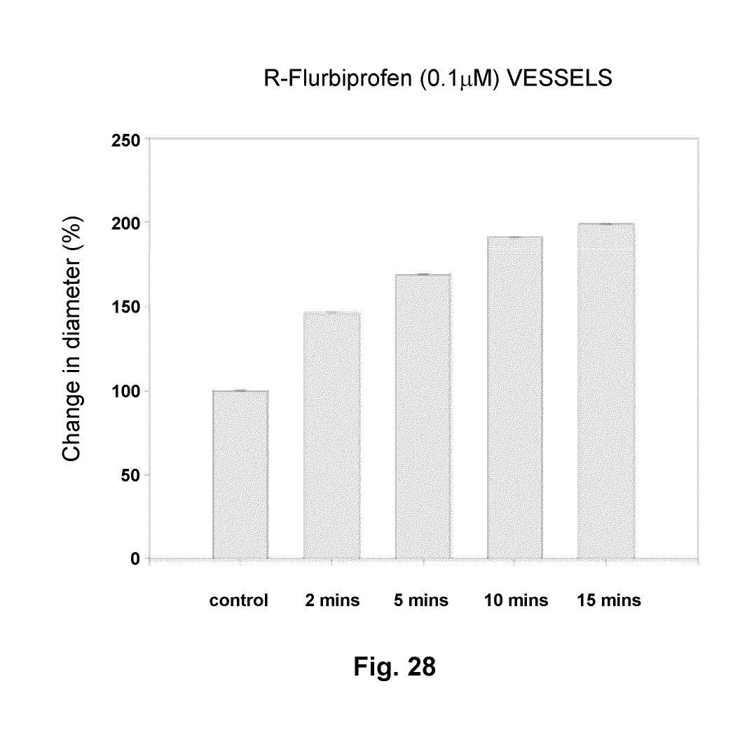

For this purpose cells were grown on silicone substrate. On the day of the experiment the cells were rinsed with HEPES-buffed physiological solution, and left in this solution for 20 min at room temperature. N-phenylanthranilic acid, flufenamic acid or R-flurbiprofen was added in increasing concentrations by fluid exchange over pericytes grown on silicone substrates. Pericytes were exposed for 10 minutes to each new concentration of N-phenylanthranilic acid, flufenamic acid or R-flurbiprofen. The concentrations of N-phenylanthranilic acid were 100 micromole/L, 300 micromole/L and 1000 micromole/L. The concentrations of flufenamic acid were 30 micromole/L, 50 micromole/L, 100 micromole/L and 300 micromole/L. The concentrations of R-flurbiprofen were 0.1 micromole/L, 0.3 micromole/L, 1 micromole/L and 3 micromole/L. See FIGS. 8, 9, and 10.

### EXAMPLE 7

### Effect of Non-Steroidal Anti-Inflammatory Drugs on Aortic Smooth Muscle Cells

It has been reported—that NSA/Ds of the fenamate family, which included mefenamic acid, niflumic acid and flufenamic acid, activated Ca²⁺-activated potassium channels [13,14]. We examined the possibility, using patch-clamp electrophysiology, that other NSAIDs may also activate Ca²⁺-activated potassium channels in aortic smooth muscle cells, since not all NSAIDs have been shown to cause significant increases in blood pressure. Furthermore, we used the enantiomers of flurbiprofen to separate the cyclo-oxygenase mediated effects from those related to potassium channel activation in organ bath experiments where we recorded constrictor responses of the aorta to phenylephrine. Our additional experimentation using femoral arteries confirmed our observations in the aorta, that a class of K⁺ channels in smooth muscle cells which are activated by both Ca²⁺ and ATP have a major role in regulating tension in the vessel wall.

### EXAMPLE 8

### Preparation of an Isolated Retina

In order to conduct the screening assay, the whole retina is obtained from an eye using the following procedure:

the eyes are enucleated immediately after the sacrifice of an animal and then the eyes are placed in small tubes containing a physiological buffer solution and stored immediately on-ice. The eyes can be transported this way over a long distance if the donor animal is some distance from the laboratory in which the screening for diagnostic compounds is performed.

The enucleated eye is placed in a shallow dish (e.g. Petri dish) and the dish containing the eye is placed on the stage of a binocular dissecting microscope in order to perform the dissection to remove the retina.

An incision is made to the outside of the eye at the position of the ora serrata so that the incision is continuous around the circumference of the eye so as to allow the anterior segment of the eye to be separated carefully from the posterior segment. The anterior segment so removed contains the cornea, iris, ciliary body and crystalline lens. The iris and ciliary body, including the ciliary muscle, are peeled from the vitreous humor that remains in the posterior segment. The crystalline lens is gently separated from the vitreous body remaining in the posterior segment of the eye.

The vitreous humor is gently separated from the posterior pole and removed from the posterior segment. This dissection leaves the retina in its usual position aligned to the interior surface of the posterior segment. The posterior segment forms a posterior eye-cup that contains the retina aligned in its normal physiological position. This posterior eye-cup and retina is then rinsed gently with a physiological buffer solution.

The posterior eye-cup and retina is fixed to a shallow dish (e.g. Petri dish) using an adhesive agent. This adhesive agent can be cyanocrylate glue (“Super-glue”) or 2-part resin (“Araldite”) or high viscosity vacuum grease or silicone (“Sylgard”). The adhesive agent allows the posterior eye-cup and retina to be firmly stabilised on the bottom of the shallow dish. This configuration creates a small reservoir of fluid above the retina which is in the position normally occupied by the vitreous humor in the living eye.

The posterior eye-cup and retina so fixed to the shallow dish is kept moist with physiological buffer solution. The dish containing the posterior eye-cup and retina is positioned on the stage of a microscope which is fitted with a digital camera to record high magnification images of the retina in the posterior eye-cup preparation. The microscope used by the inventors were a dissecting or operating microscope.

### EXAMPLE 9

### The Effect of Vasoactive Compounds on the Isolated Retina

The vasoactive compound is tested for activity by introducing it to the small reservoir contained within the posterior eye-cup. In this way the vasoactive compound contacts the retina from the vitreous side. Different vasoactive compounds and rinsing solution of physiological buffer are applied sequentially to the posterior eye-cup and retina preparation.

Images of the retina are recorded using the digital camera attached to the optical viewing pathway of the microscope. The images of the retina are analysed by measuring the change in dimension of the retinal blood vessels, which include the capillary vessels and the larger arterioles. Preferably, the dimension that is measured is the cross-sectional diameter of the blood vessel.

There is usually a small volume of blood remaining in the blood vessels following the enucleation and dissection of the eye to produce the posterior eye-cup and retina preparation. This small volume of blood renders the blood vessels to be visible in the images recorded by the digital camera.

An alternative method of rendering the blood vessels visible is to perfuse the retinal blood vessels with sterile Evans Blue solution after the posterior eye-cup and retina preparation is fixed to the shallow dish for viewing and imaging by the microscope. The method of perfusing the Evans Blue is to first produce a pointed end on a glass microcapillary tube utilising a microelectrode pulling machine. The outside diameter of this pointed end is between 5 and 30 microns. The glass microcapillary tube is filled with the Evans Blue solution. The untreated end of the glass microcapillary tube (opposite to the point) is inserted into a commercially available microelectrode holder that has a venting port. The venting port is connected to a syringe pump using flexible tubing. A larger retinal blood vessel near the optic nerve head is cannulated using the pointed end of the glass microcapillary tube. The syringe pump is then used to perfuse the Evans Blue solution into this cannulated retinal blood vessel. After several minutes the Evans Blue solution fills the larger retinal blood vessel and the smaller branching vessels. The glass microcapillary tube is removed from the retinal blood vessel after Evans Blue solution has filled numerous branches of the retinal blood vessels that emanate from the larger retinal blood vessel that was originally cannulated.

Another alternative method of rendering the retinal blood vessels visible is to use the pointed glass microcapillary tube to cannulate the blood vessels from the outside of the eye prior to dissection. In this alternative method the Evans Blue is perfused into the large blood vessels that enter the eye. After several minutes of perfusion, the glass microcapillary tube is removed from the blood vessel and the eye is enucleated and dissected according the above procedure to prepare the posterior eye-cup and retina.

Another alternative method of rendering the blood vessels visible is to anaesthetise the animal and introduce a standard blood vessel cannula into the femoral artery. The cannula is attached to a syringe pump which is used to perfuse sterile Evans Blue into the femoral artery. The Evans Blue introduced into the femoral artery is pumped through the retinal circulation by the beating heart of the anaesthetised animal. The Evans Blue is perfused into the femoral artery for several minutes. After that time the cannula is withdrawn from the femoral artery and the animal is sacrificed, and the eye is enucleated and dissected according the above procedure to prepare the posterior eye-cup and retina.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

What is claimed is:

1. A method of determining or identifying a compound that modulates the function of a blood vessel in an isolated retina-comprising contacting an isolated retina with a test compound and determining a change in the contractile state of said blood vessel, wherein said change indicates that the compound modulates blood vessel function.

2. A method of determining or identifying a compound that modulates the contractile state of a blood vessel in an isolated retina comprising:
providing an isolated retina;
(ii) contacting a test compound with said isolated retina; and
determining a distortion of said blood vessel, wherein said distortion indicates that said compound modulates the contractile state of a blood vessel in an isolated retina.

3. The method of determining or identifying a compound of claim 1, wherein the isolated retina is fixed to a support.

4. The method of determining or identifying a compound of claim 1, wherein the retina is a whole retina.

5. The method of determining or identifying a compound of claim 1, wherein the contractile state of a blood vessel is determined visually.

6. The method of determining or identifying a compound of claim 1, wherein the size, dimension or volume of the blood vessel is determined.

7. The method of determining or identifying a compound of claim 2, wherein the method further comprises:
washing the isolated retina with a suitable buffer or aqueous solvent that is not damaging to the integrity of the retina or contractile function of the retinal blood vessels for a time and under conditions sufficient to remove the compound or reduce its activity to a level that does not affect blood vessel contraction.

8. The method of determining or identifying a compound of claim 1, further comprising:
determining a distortion in the retinal blood vessel;contacting the retinal blood vessel with a second test compound; and determining a distortion in said retinal blood vessel, wherein said distortion indicates that said compound modulates the contractile state of the retinal blood vessel.

9. A method of diagnosing impaired retinal blood vessel function in a subject comprising administering to the subject a pharmaceutically acceptable amount of a compound that modulates blood vessel function under conditions sufficient to modify the contractile state of a blood vessel, wherein said compound is identified by a method comprising determining a change in the contractile state of a blood vessel in an isolated retina in the presence of said compound, wherein said change is indicative that the compound modulates blood vessel function, and detecting a change in the contractile state of the subject's retinal blood vessels.

10. A method of diagnosing impaired retinal blood vessel function in a subject, the method comprising:
administering to the subject a pharmaceutically acceptable amount of a compound that modulates the contractile state of a blood vessel in an isolated retina wherein said compound is identified by a process comprising determining a change in the contractile state of a blood vessel in an isolated retina in the presence of said compound, wherein said change is indicative that the compound modulates retinal vessel function; and
detecting a distortion of a blood vessel in the retina of the subject,
wherein a slow or unsubstantial dilation or constriction of the retinal vessel indicates retinal vessel damage.

11. The method of diagnosing impaired retinal blood vessel function of claim 9, wherein size, dimension or volume of the blood vessel is determined.

12. The method of diagnosing impaired retinal blood vessel function of claim 9, wherein the method comprises contacting the subject's eye with an effective amount of at least one compound selected from the group consisting of:
pituitary adenylate cyclase-activating polypeptide (PACAP), vasoactive intestinal polypeptide (VIP), a compound having activity on phospholipase C (PLC), a compound having activity on protein kinase A (PKA), a compound having activity on ion-channel hyperpolarisation channels, and a non-steroidal anti-inflammatory drug (NSAID), or a homologue, analogue or derivative thereof.

13. The method of diagnosing impaired retinal blood vessel function of claim 9, wherein the NSAIDs is selected from the group consisting of aspirin, pyrazolones, fenamate, diflunisal, acetic acid derivatives, propionic acid derivatives, oxicams, fenamates such as mefenamic acid, meclofenamate, the group consisting of: phenylbutazone, diflunisal, diclofenac, Voltaren, indomethacin, sulindac, N-phenylanthranilic acid, etodolac, ketorolac, nabumetone, tolmetin, ibuprofen, fenoprofen, flurbiprofen, carprofen, ketoprofen, naproxen, piroxicam, indomethacin and flufenamic acid, and a derivative thereof.

14. The method of diagnosing impaired retinal blood vessel function of claim 9, wherein the NSAID is N-phenylanthranilic acid or flufenamic acid or flurbiprofen.

15. The method of diagnosing impaired retinal blood vessel function of claim 9, wherein the NSAID is flurbiprofen in the R-isomer form, or S-isomer form.

16. A method of treating a subject having impaired retinal blood vessel function comprising administering to the subject an amount of a pharmaceutical composition comprising a compound that modulates retinal blood vessel function and a pharmaceutically acceptable carrier, diluent or excipient, wherein said compound is identified by a process comprising determining a change in the contractile state of a blood vessel in an isolated retina in the presence of said compound, wherein said change is indicative that the compound modulates retinal blood vessel function.

17. The method of treating a subject according to claim 16, wherein said compound is identified by:
providing an isolated retina;
contacting a test compound with said isolated retina; and
determining a distortion of said blood vessel, wherein said distortion indicates that said compound modulates the contractile state of a blood vessel in an isolated retina.

18. A method of treating a subject having impaired retinal blood vessel function comprising administering to the subject an amount of a pharmaceutical composition comprising a compound that modulates retinal blood function and a pharmaceutically acceptable carrier diluent or excipient, wherein said compound is selected from the group consisting of:
pituitary adenylate cyclase-activating polypeptide (PACAP), vasoactive intestinal polypeptide (VIP), a compound having activity on phospholipase C (PLC), a compound having activity on protein kinase A (PKA), a compound having activity on ion-channel hyperpolarisation channels, and a non-steroidal anti-inflammatory drug (NSAID), or a homologue, analogue or derivative thereof.
